(19) United States
(12) Patent Application Publication
(10) Pub. No.: US 2022/0252586 A1
(43) Pub. Date: Aug. 11, 2022

(54) COMPOSITIONS AND METHODS FOR DETECTING AND DEPLETING SAMPLE INTERFERENCES

(71) Applicant: Veravas, Inc., Charleston, SC (US)

(72) Inventors: Joshua Caine Soldo, Prior Lake, MN (US); Scott Douglas Bergmann, River Falls, WI (US); Eric Nelson, Charlston, SC (US)

(21) Appl. No.: 17/636,012

(22) PCT Filed: Jun. 25, 2020
(86) PCT No.: PCT/US2020/039503

§ 371 Date: Feb. 16, 2022

Related U.S. Application Data
(60) Provisional application No. 62/866,318, filed on Jun. 25, 2019.
(60) Provisional application No. 63/006,630, filed on Apr. 7, 2020.

(51) Int. Cl. G01N 33/543

(52) U.S. Cl. CPC G01N 33/54326; G01N 33/54393

(57) ABSTRACT

The present specification provides methods to prepare reagents and microparticulate binding surfaces with specificity to anti-streptavidin interference and anti-biotin interference. Also disclosed is how to use reagents and microparticulate binding surfaces to block, deplete or reduce the concentration of anti-streptavidin interference and anti-biotin interference in samples below the assay blocking threshold (ABT) prior to a diagnostic test.

### CROSS-REFERENCE TO RELATED APPLICATIONS

This application claims the benefit of priority to U.S. Provisional Application Nos. 63/006,630, filed Apr. 7, 2020, and 62/866,318, filed Jun. 25, 2019, the entire contents of which are each incorporated by reference herein.

### BACKGROUND

Every nine minutes, someone dies due to an incorrect or delayed diagnosis [1]. Physicians rely on diagnostic tests to guide treatment, but 2% or more of tests can be inaccurate due to multiple interferences in blood or urine (e.g., biotin in blood tests) [2].

Biotin, also known as Vitamin B7, Vitamin H and Coenzyme R, is a water-soluble vitamin often found in high doses in over the counter (OTC) dietary supplements, multi-vitamins, and prenatal vitamins. Biotin is marketed for health & beauty including hair, skin and nail growth, as well as weight loss. It is also given to patients at high therapeutic doses to treat certain medical conditions such as multiple sclerosis. However, biotin can significantly interfere with certain lab tests and cause incorrect test results which may go undetected and may lead to misdiagnosis or delayed treatment [3-13].

In 2017, the FDA issued a safety warning as the number of adverse events has been related to inaccurate test results and biotin supplementation [14]. On Jun. 13, 2019 the FDA released a notification of a draft guidance document on “Testing for Biotin Interference in In Vitro Diagnostic Devices” [15].

In vitro diagnostic (IVD) companies are actively working to redesign or reformulate their tests to mitigate biotin interference, or to increase biotin interference thresholds, such that it takes much higher biotin concentrations to interfere with the test. However, these biotin-based tests are still susceptible to secondary interference mechanisms associated with biotin or biotin use by patients, or anti-biotin interference [16-17], as well as an interference mechanism associated with the use of streptavidin in the test design to capture biotin which has been conjugated to antibodies, proteins or antigens, or anti-streptavidin interference [18-26].

Anti-biotin and anti-streptavidin antibodies & proteins can significantly interfere with certain lab tests and cause incorrect test results. Similar to biotin interference which causes a decreased test signal and false low or false high patient results depending on the assay design and format, anti-biotin and anti-streptavidin interference also results in a decreased test signal but via different mechanisms, and therefore they can be mistaken for biotin interference [16-26].

Although the FDA has recently provided guidance that IVD companies should test for biotin interference at concentrations up to 1200 ng/mL consistent with the recommendations in the Clinical & Laboratory Standards Institute (CLSI) standard, and to reflect current trends in biotin consumption, the FDA has not yet issued a safety warning for adverse events related to inaccurate test results due to human anti-biotin or human anti-streptavidin interference [15]. While human anti-streptavidin and human anti-biotin interference have been reported in the literature, it is difficult to detect and confirm these specific interference mechanisms or differentiate them from biotin interference.

Sample pre-treatment with a binding surface (i.e. magnetic beads, non-magnetic beads, nanoparticles, microtiter plate/well, cuvette, slide, sensor, chip, rod, filter, membrane, tube, or any other solid phase used to process samples) immobilized or covalently conjugated to capture moieties or interference-specific targets can be used to deplete, enrich and/or characterize sample interferences or biomarkers prior to a diagnostic test to improve the quality and accuracy of test results [27].

### SUMMARY

A large proportion of IVD assays make use of streptavidin-biotin binding. These assays are subject to heterophilic interference from free biotin and agents that compete or otherwise interfere with the binding between assay reagents and streptavidin or biotin, including anti-streptavidin antibodies and anti-biotin antibodies. Substances that interfere with the binding between assay reagents and streptavidin or biotin are referred to herein as anti-biotin or anti-streptavidin whether or not the substance is an antibody. Disclosed herein are reagents (beads) that can be used 1) to detect or quantitate these different types of interference, and 2) to remove or deplete the interfering substances that may be present in assay reagents, samples, or reaction mixtures, so that more accurate assay results may be obtained. Methods for making and using these reagents are also provided.

Some of the herein disclosed reagents comprise nanoparticles which have been coated with streptavidin, to form a streptavidin bead. In some embodiments, some or all of the streptavidin has been covalently conjugated to the nanoparticle. In some embodiments the nanoparticle is magnetic, to facilitate separation of the bead from storage solution and treated samples, assay reagents, etc. In some embodiments the nanoparticle may or may not be magnetic and separation of the bead from storage solution and treated samples is accomplish by sedimentation (such as centrifugation) or filtration. Other embodiments comprise free (or soluble) streptavidin. In either type of embodiment, free or coated bead, the streptavidin is saturated (or quenched), preferably saturated, with minimal excess biotin, so that the streptavidin cannot bridge between biotinylated assay reagent molecules, and become a source of heterophilic interference. By saturated, it is meant that all accessible biotin binding sites on the streptavidin are occupied by biotin. The free biotin-saturated streptavidin is suitable for use as an anti-streptavidin heterophilic interference blocking reagent that, for example, can be added to (present in) an assay reaction mixture. The biotin-saturated streptavidin coated bead is suitable for use as an anti-streptavidin heterophilic interference cleaning reagent that, for example, can be added to a biological fluid or extract to be assayed (a sample) and then removed prior to the sample being added to the assay reaction mixture. In some uses, a cleaning reagent can be added to assay reagents or partial assay reaction mixtures and removed prior to completing the assay reaction mixture and starting the assay reaction.

Embodiments utilizing streptavidin are described throughout this disclosure. However, further embodiments comprising alternatives, such as avidin, deglycosylated avidin (neutravidin), CaptAvidin, monomeric avidin, are also contemplated. Natural and recombinant versions of streptavidin, and its alternatives, are also contemplated. These reagents may be referred to as means for binding biotin or means for binding anti-streptavidin interference.

Embodiments utilizing biotin to quench or saturate streptavidin active biotin binding sites are described throughout this disclosure. However, further embodiments using biotinylation agents, such as Biotin-PEGₙ-COOH or Biotin-PEGₙ-CH₃ or Biotin-PEGₙ-OH or other Biotin-R-(non-reactive end chemistry) where R is a carbon chain or ring structure, are also contemplated. Biotin and these modified forms of biotin may be referred to as means for binding to streptavidin or means for binding anti-biotin interference.

In further embodiments, the streptavidin-coated bead, biotin-saturated streptavidin-coated bead, streptavidin or quenched streptavidin is modified by conjugation to one or more additional capture moieties for the removal of other heterophilic or cross-reactive interferences (in addition to anti-streptavidin interference). In one aspect of these embodiments the additional capture moiety is biotin conjugated to the biotin-saturated streptavidin-coated bead and is additionally suitable for use as an anti-biotin heterophilic interference cleaning reagent. In further aspects of these embodiments the additional capture moiety is ruthenium (an element); luminol, acridinium ester, ABEI or cyclic ABEI (like biotin, small organic molecules); or a protein, such as a signal-generating enzyme, for example, alkaline phosphatase or horse-radish peroxidase; streptavidin; an antibody, for example, an antibody from a non-human species; or an antigen. In still further aspects, the capture moiety can be any non-antibody peptide or protein. In all these instances, the additional capture moiety makes the streptavidin-coated bead or biotin-saturated streptavidin-coated bead suitable for use as a cleaning reagent for removing or depleting heterophilic or cross-reactive interference associated with the conjugated molecule. Some embodiments specifically include one or more capture moieties. Some embodiments specifically exclude one or more capture moieties. For example, in some embodiments the additional capture moiety is not biotin.

Several chemistries are available to accomplish the conjugation to streptavidin (soluble, or bead-bound, biotin saturated or not). Conjugation may proceed using an amine reactive reagent to form a bond to the primary amines of streptavidin, typically an ester, for example an NHS-modified compound or protein. Alternatively, the primary amines of streptavidin may be thiolated. Standard thiolation reagents are known in the art, but include Succinimidyl trans-4-(maleimidylmethyl)cyclohexane-1-Carboxylate (SMCC) and Succinimidyl 3-(2-Pyridyldithio)Propionate (SPDP). Conjugation can then be performed using a thiol- or sulfhydryl-reactive reagent, such as a maleimide-modified compound or protein. In another alternative, the primary amines of streptavidin are reacted with maleimide using standard ester-maleimide heterobifunctional crosslinkers. Conjugation can then be performed using a thiol- or sulfhydryl-modified (or containing) compound or protein. These chemistries and associated reagents may be referred to as means for conjugation, and the reactions themselves as a step for conjugation.

Typically, conjugation of an additional capture moiety to the streptavidin-coated bead, biotin-saturated streptavidin-coated bead, streptavidin or quenched streptavidin involves use of a heterobifunctional linker. The functional group at one end of the linker to form a covalent attachment to the streptavidin and the functional group at the other end to form a covalent attachment to the additional capture moiety. The chemistry of particular functional groups is discussed herein below. In some embodiments capture moiety attached to a linker may be commercially available. Some embodiments specifically include a particular functional group or set of functional groups. Some embodiments specifically exclude a particular functional group or set of functional groups. In some embodiments, the central portion of the heterobifunctional linker comprises polyethylene glycol (PEG) or polyethylene oxide (PEO). In aspects of this embodiment the linker may comprise multiple units of PEG of PEO, for example, PEGₙ or PEOₙ where n is any integer from 1 to 36. In further aspect the PEG linker can be branched or dendritic, such as monodisperse PEGs, trifunctional PEGs, 4-arm PEGs, 8-arm PEGs, heterobifunctional PEGs, homobifunctional PEGs, instead of linear monofunctional PEGs. Other linkers are disclosed herein below.

Various ways of conjugating biotin to biotin-saturated streptavidin are disclosed herein below. However, any other capture reagent may be analogously conjugated to streptavidin (biotin saturated or not, bead-bound or not).

In alternative embodiments the additional capture moiety is not covalently attached, but is attached using a biotin linker. In some embodiments the additional capture moiety is biotin and the linker has a biotin molecule at each end, for example biotin-PEGₙ-biotin. In such embodiments, the bis-biotin linker is added as a minor percentage of the biotin used in the streptavidin saturation procedure (to avoid bridging between beads). Thus, the biotin at one end binds to streptavidin while the biotin at the other end is free to serve as a capture moiety. In other embodiments, a linker with biotin at one end and any other capture moiety at the other end is used, for example, biotin-(PEO)n-ruthenium. In further embodiments, two or more different capture moieties can be introduced via this approach, such as co-coating streptavidin with biotin-(PEO)n-ruthenium and biotin-(PEO)n-alkaline phosphatase. In these embodiments a potentially greater proportion of the biotin used in the streptavidin saturation procedure can be the capture moiety-linked biotin, as bridging between beads should not be an issue; however, steric considerations based on the size of the capture moiety and length of the linker can be a factor limiting the proportion.

Some embodiments are methods of mitigating interference in a liquid biological sample. Other embodiments are methods of reducing interference in a diagnostic assay. In some embodiments biotin-saturated streptavidin (biotin-quenched streptavidin; QSAv) is combined with a liquid biological sample to form a mixture which is mixed to facilitate binding of the interference to the streptavidin so as to block or reduce the interference. Some embodiments further comprise conducting a diagnostic assay. In some embodiments, the combining and mixing take place prior to the analytic phase of the assay. As used herein, term “analytic phase of an assay” commences when the sample is mixed with the reagents to capture or detect the analyte, and/or generate signal indicating or quantitating the presence of the analyte, and continues through measurement of the signal.

In other embodiments (for mitigating or reducing interference), a particle comprising streptavidin, biotin-quenched or not, is combined with a liquid biological sample to form a mixture which is mixed to facilitate binding of the interference to the streptavidin and the particle is separated from the sample so as to remove or reduce the interference. Some embodiments further comprise conducting a diagnostic assay. In some embodiments, the combining, mixing, and separating take place prior to the analytic phase of the assay. In one aspect of these methods, the particle is magnetic, and separating the particle from the sample comprises exposing the mixture to a magnet and collecting the liquid sample. In further aspects, the sample is not diluted, and there is little or no sample loss.

In some embodiments of these methods for mitigating or reducing interference, the sample is used in a sandwich immunoassay. In other embodiments the sample is used in competitive immunoassay.

Some embodiments are methods of making a quenched streptavidin. Some of these embodiments comprise exposing the streptavidin to a minimal molar excess of free biotin. In one aspect this can comprise metered addition to combine a biotin solution with a streptavidin solution. Some of these embodiments comprise washing the quenched streptavidin with hot buffer. In one aspect this can comprise diafiltration. Some of the embodiments comprise blocking the streptavidin to avoid formation of aggregates. Some embodiments comprise conjugating an additional capture moiety to the quenched streptavidin. Some embodiments comprise quenched streptavidin made by any of these methods.

Some embodiments are methods of making a particle-conjugated streptavidin. Some of these embodiments comprise exposing the particle-conjugated streptavidin to a minimal molar excess of free biotin. In one aspect this can comprise metered addition to combine a biotin solution with a particle-conjugated streptavidin suspension. Some of these embodiments comprise washing the quenched streptavidin with hot water. In one aspect this can comprise magnetic separation of particles. In other aspects this can comprise separation of the particles by filtration or sedimentation. Some embodiments comprise conjugating an additional capture moiety to the particle-conjugated streptavidin, biotin-quenched or not. Some embodiments comprise particle-conjugated streptavidin, biotin-quenched or not, made by any of these methods.

### BRIEF DESCRIPTION OF DRAWINGS

FIG. 1 depicts the size distribution of biotinylated 100BS streptavidin beads. This data demonstrates a uniform size single peak of 883.9 nm with a 12.2% polydispersity index.

FIG. 2A-B depicts the size distribution of (2A) biotinylated 100BS streptavidin beads after 30 minutes incubation with streptavidin. Bead aggregation occurred with peaks at 1,441.3 nm and 6,641 nm with a polydispersity index of 173.3% and (2B) biotinylated 100BS streptavidin beads after 4 hours incubation with streptavidin. Bead aggregation occurred with peaks at 1,512.6 nm and 14,536 nm with a polydispersity index of 242.8%.

FIG. 3 depicts the size distribution of biotinylated 100BS streptavidin beads after overnight incubation with monoclonal anti-biotin conjugate antibody. Bead aggregation occurred with a peak at 2,148 nm with a polydispersity index of 316.2%

FIG. 4A-C depicts (4A) SEC-HPLC standard curve for the anti-biotin antibody. The data point indicated by the arrow corresponds to the Peak Area and anti-biotin antibody remaining (μg/mL) in the sample after pre-treatment with the biotinylated 100BS streptavidin-beads to deplete anti-biotin antibody. 4B-C depicts SEC-HPLC analysis of the anti-biotin antibody (4B) pre- and (4C) post-depletion with the biotinylated 100BS streptavidin-beads. The peak area decreased from 1,384 to 318, and the anti-biotin concentration decreased from 205 μg/mL to 44.62 μg/mL, after depletion of the anti-biotin antibody.

FIG. 5A-D are chromatograms before and after treatment from HPLC-SEC depletion assays using biotinylated 100BS streptavidin-beads. 5A shows absence of depletion of affinity-purified goat IgG by the beads. 5B shows absence of depletion of biotinylated affinity-purified goat IgG by the beads. 5C shows depletion of goat anti-biotin Ab by the beads. 5D shows depletion of goat anti-streptavidin by the beads. In all cases the profile for untreated and treated are indicated by labeled arrows.

FIG. 6 depicts the amount of serum parathyroid hormone detected by ELISA with various interferents and with and without treatment with biotinylated 100BS streptavidin-beads. None—no interferent; Biotin->250 ng/mL; Anti-Biotin IgG—16.5 μg/mL of Ab; Anti-SAv IgG 16.5 μg/mL of Ab; Anti-Biotin IgG/SAv IgG—8.25 μg/mL of each Ab.

FIG. 7 depicts the apparatus for metered addition of biotin to streptavidin in the saturation procedure.

FIG. 8 depicts the apparatus for diafiltration, washing, and concentration of the biotin-saturated streptavidin.

### DETAILED DESCRIPTION

Despite existing approaches to the identification or depletion of compound causing assay interference, a clinical need remains for fast and easy-to-use product solutions to detect and mitigate anti-biotin and anti-streptavidin interference in patient samples. Such product solutions would also facilitate prevalence studies to help clinicians and laboratory medicine professionals better understand which patients and patient populations are at greatest risk for these interferences.

There is also a clinical need to mitigate anti-streptavidin interference in patient samples by using a blocking reagent specific for anti-streptavidin interference in the assay formulation. This product solution would also have the least impact on laboratory workflow as anti-streptavidin interference would be mitigated by the diagnostic test design.

Immunoassays are subject to interference that can lead to the reporting of false high or low levels of the analyte being assayed. One type of interference relates to signal generation and observation. These include factors such as turbidity, hemolysis, quenching, and inhibition of signal generating enzymes. In general, these interferences are directly observable or can be tested for without specialized reagents. The herein disclosed embodiments do not address such signal generation/observation interferences, and general reference to interference herein does not include such interference.

Another type of immunoassay interference relates to capture and physical detection of analyte. These include interferences that inhibit interaction between the analyte and capture or detection reagents, or cause association of capture and detection reagents without regard for the presence (or absence) of the analyte. This type of interference is termed heterophilic interference; as used herein ‘interference” should be understood to mean heterophilic interference unless context dictates otherwise. The herein disclosed embodiments address various specific heterophilic interferences. In general heterophilic interferences are not directly observable, nor can their presence be readily demonstrated with standard assay reagents. Some of the herein disclosed embodiments can be used to demonstrate the presence of, or to quantitate, a particular heterophilic interference. Heterophilic interferences include biotin, anti-biotin, anti-streptavidin, and anti-xenoantibody interferences, as well as interferences that bind to components of an assays signal generation system (enzymes, fluors, etc.). Anti-xenoantibody interferences include human antibodies recognizing mouse, rat, rabbit, sheep, bovine, and or goat immunoglobulins.

Another type of immunoassay interference relates to cross-reactive antibodies. Cross-reactivity between antigens occurs when an antibody directed against one specific antigen is successful in binding with another, different antigen. In other words, cross-reactivity involves the binding by an antibody to an antigen other than its immunogen. This can be particularly problematic, for example, in an immunoassay intended to detect antibodies recognizing antigens from a particular strain of bacteria or virus. Such assays are commonly used to determine if the subject has been exposed to (infected by) the pathogen or agent in question. If the subject has been previously exposed to a related strain they may have antibodies that will cross-react with antigen from the strain that the assay is intended to detect, and thus generate a false positive result.

As used herein “immunoassay” generally refers to an assay in which detection or quantitation of analyte employs the use of an antibody (or an antigen binding fragment or derivative thereof) that specifically binds to the analyte. However, it is also possible to design assays in which a non-antibody agent which can specifically bind the analyte is used analogously to an anti-analyte antibody. In some embodiments, the non-antibody agent which can specifically bind the analyte is an aptamer or a molecularly imprinted polymer. Thus, in some embodiments, “immunoassay” can encompass assays in which a non-antibody agent provides the analyte-specific binding activity typically supplied by an antibody. Various embodiments specifically include or exclude antibodies or non-antibody agents as the analyte-specific binding activity. Some embodiments specifically include or exclude an aptamer or a molecularly imprinted polymer. Immunoassay can be divided into classes based on the technology and physical arrangement of components; the assay format. One class involves combining sample (potentially containing analyte) with detection and/or signal generating reagents in a container, such as a microtube or microtiter plate, where the assay reaction takes place. Reagents can be added to or removed from the container in the course of the assay. (In some variations some portion of the assay components are removed from the initial container and added to a 2nd container in which the assay proceeds.) Such assays shall be referred to herein as “pot” assays. In another class, some of the detection and/or signal generating reagents are fixed to specific regions of a solid substrate or matrix, for example a membrane. The sample (potentially containing analyte) is applied to a particular location of the apparatus containing the solid substrate or matrix and encounters the fixed reagents by moving, for example by lateral flow, into and often through the region within in which the reagents are fixed. Additional assay reagents will move with the mobile phase. Such assays are referred to herein as “zonal” assays. From the point at which sample is added to the container in which the assay reaction with take place for a pot assay, or the point at which the sample is add to the particular location of the apparatus containing the solid substrate or matrix for a zonal assay, through measurement of the generated signal, is referred to as the analytic phase of the assay. In many embodiments, the herein disclosed interference cleaning or blocking reagents are added to, and with respect to cleaning reagents, removed from the sample prior to the analytic phase; that is, in these embodiments, the interference reducing reagents are used as a “pre-treatment”.

Patients who consume high doses of biotin for health & beauty (5,000 to 20,000 mcg per day) or therapeutically (100,000 to 300,000 mcg per day) can have high circulating concentrations of biotin in their blood up to 1,000 ng/mL or greater depending on how long it has been since biotin ingestion, the patient-specific biotin clearance time, and if the patient has kidney disease or poor kidney function which may impair biotin clearance and increase circulating levels of biotin. If a patient's free biotin has not yet cleared below the test-specific biotin interference threshold prior to drawing a blood, serum or plasma sample, or prior to collecting a urine sample, any biotin in the sample greater than the test-specific biotin interference threshold will compete for and bind to the anti-biotin capture moiety (i.e. streptavidin, avidin, neutravidin, monomeric avidin, CaptAvidin, or antibody, antibody fragment/Fab/F(ab)′2, aptamers, and molecular imprinted polymers with specificity to biotin) and subsequently interfere with the binding of the biotinylated antibody, protein or antigen used in the assay formulation. This will result in a false low assay signal, and depending on the assay format a false low dose (sandwich assay) or false high dose (competitive inhibition assay).

Streptavidin is a ˜52,000-55,000 KDa protein and is composed of 4 identical polypeptide chains. As used herein, monomeric streptavidin refers to non-aggregated streptavidin protein, and not to dissociated streptavidin polypeptide chains. The binding of biotin to streptavidin (reported variously as 10⁻¹⁴ or 10⁻¹⁵ mol/L in the literature) is one of the strongest non-covalent interactions known in nature. Recombinant streptavidin is a suitable tool for enabling universal test systems in immunology and molecular diagnostics, and it is commonly used in diagnostic tests such as immunoassays to capture biotinylated antibodies, protein, and antigens, or to attach various biomolecules to one another or onto a solid support such as such as microplates, beads, and microarrays. The use of streptavidin also enables assay developers to take advantage of the proven anti-biotin delayed capture assay format for improved assay kinetics, precision and sensitivity, while facilitating shorter assay incubation times and faster turn-around times (TAT) for STAT assays.

If a sample contains interference with specificity to streptavidin, the anti-streptavidin interference can bind to streptavidin or its polypeptide chains and sterically block or impair conjugated biotin from binding to streptavidin's biotin binding sites. If streptavidin can no longer freely bind the biotinylated antibody, protein, or antigen used in the test design or assay format, just like biotin interference, anti-streptavidin interference will result in a false low assay signal and can result in a false low dose (sandwich assay) or false high dose (competitive inhibition assay). Similarly, if a sample contains interference with specificity to biotin, the anti-biotin interference can bind to the biotin and sterically block or impair conjugated biotin from binding to streptavidin's biotin binding sites. If biotin from a biotinylated antibody, protein, or antigen, etc., used in the test design or assay format, anti-biotin interference will result in a false low assay signal and can again result in a false low dose (sandwich assay) or false high dose (competitive inhibition assay). Some embodiments address anti-streptavidin interference. Some embodiments address both anti-streptavidin and anti-biotin interference.

Whereas the biotin-streptavidin interaction is commonly used in the capture portion of an immunoassay mechanism, heterophilic interference can also arise through interaction with common detection components. Such components can include fluors such as fluorescein or the element ruthenium; chemiluminescents such as luminol, acridinium ester, ABEI, and cyclic ABEI; bioluminescents, such as luciferin; and enzymes such as alkaline phosphatase or horseradish peroxidase. Interferences that bind to these signal generating molecules can cause cross-linking between detection antibodies (or other detection reagents) that are bound to analyte and those that are not to result in a false high signal. Some embodiments address both anti-streptavidin and anti-signal generating molecule interference.

Immunoassays typically make use of antisera, polyclonal antibodies, or monoclonal antibodies from non-human species. Sera or other assay samples may contain interferences that recognize these xenoantibodies, sometimes termed human anti-animal antibodies (HAAA). In particular, humans produce antibodies against a wide variety of animal species. Commonly, those are the species with which the most interaction happens such as mice, cows, horses, dogs, cats, goats, rabbit, and sheep. Among those, antibodies, particularly IgG, from mice, goats, rabbit, and sheep are very commonly used in clinical immunochemical assay systems. However, similar heterophilic interferences can arise in samples from non-human subjects. Such anti-antibody interferences can cause cross-linking between capture and detection antibodies in the absence of bound analyte, or cross-linking between detection antibodies that are bound to analyte and those that are not, to result in a false high or false low signal. Some embodiments address both anti-streptavidin and anti-xenoantibody interference.

There are two modes of addressing immunoassay interference, blocking and cleaning. As used herein, a blocking reagent is present in the assay reaction and, through its interaction with the interfering substance, prevents or reduces the interference. Some embodiments comprise or make use of a soluble, biotin-saturated streptavidin and are suitable for blocking anti-streptavidin interference. In some embodiments the soluble, biotin-saturated streptavidin is conjugated with a 2nd molecule subject to being bound by an interfering substance, for example, biotin, a signal generating molecule, or a xenoantibody. Embodiments comprising or making use of a soluble, biotin-saturated streptavidin conjugated with a 2nd interference target molecule are suitable for blocking both anti-streptavidin and anti-2nd molecule interference. Some embodiments specifically include one or more genera or species of 2nd interference target molecule. Some embodiments specifically exclude one or more genera or species of 2nd interference target molecule. Blocking reagents may be added during the analytic phase of the assay, or added at a pre-analytic phase and remain in the analytic phase. In some embodiments, blocking reagents can also be encountered during the analytic phase of a zonal assay, such as a lateral flow assay, or may be retained in a particular zone of such an assay. As used herein, the analytic phase of an assay refers to the temporal and/or physical portions of the assay or assay system in which analyte capture, detection, and quantitation occur.

It is noted that the terms “block” and “blocking” are used herein with more than one, though conceptually related, meanings. In the preparation of the herein disclosed reagents “blocking” etc. is used to describe obstructing or otherwise reducing the reactivity of chemically reactive sites and the effective affinity of specific and/or non-specific binding sites. This can be referred to as preparative blocking. Thus detergents and polymeric blocking reagents used herein to prevent reaction and/or non-specific binding to the core nanoparticle of the herein disclosed streptavidin coated beads, or aggregation of proteins, relate to this meaning of “block” and “blocking”. The saturation of streptavidin with biotin can also be viewed as a form of preparative blocking is which biotin is the blocking reagent. Preparative blocking should not be confused with blocking that prevents or reduces assay interference, which is a distinct function.

As used herein, a cleaning reagent is added to a serum or other biological sample—or other component of an immunoassay reaction mixture—and then removed from the sample or other component prior to mixing the components of the immunoassay reaction mixture together. That is, the cleaning reagent is used and removed in the pre-analytic phase of the assay and is not present in the analytic phase. By depleting or removing the interfering substance from the sample and/or other assay reagent(s) interference is prevented or reduced. Some embodiments comprise or make use of a biotin-saturated streptavidin that is coated onto a magnetic nanoparticle, a biotin-saturated streptavidin bead. Such biotin-saturated streptavidin beads are suitable for cleaning streptavidin interference. In some embodiments the bead's biotin-saturated streptavidin is conjugated with a 2nd molecule subject to being bound by an interfering substance, for example, biotin, a signal generating molecule, a xenoantibody, or an antigen. Embodiments comprising or making use of a biotin-saturated streptavidin bead in which the streptavidin is conjugated with a 2nd interference target molecule are suitable for cleaning both anti-streptavidin and anti-2nd molecule interference. Some embodiments specifically include one or more genera or species of 2nd interference target molecule. Some embodiments specifically exclude one or more genera or species of 2nd interference target molecule.

The biotin-saturated streptavidin beads can be magnetically separated from their storage buffer, the storage buffer removed, and then the sample or reagent to be cleaned added to the beads, so that the sample or reagent is not diluted in the cleaning process, unlike the use of a soluble blocking reagent. In other embodiments, the beads are separated from the fluid phase by filtration or sedimentation.

Tests that use streptavidin in the test design, format or formulation cannot simply use native streptavidin as a specific blocker, additive or component in the assay buffer to mitigate anti-streptavidin interference in the sample. If used as a blocker in the test, streptavidin may also compete for and bind the biotinylated antibody, protein, oligomer or antigen and result in a false low assay signal and false low dose (sandwich assay) or false high dose (competitive inhibition assay). This is particularly a concern with streptavidin since it has such a strong binding constant and affinity for biotin. While some tests can mitigate lower titers or concentrations of anti-streptavidin interference by increasing total amount or total concentration of the streptavidin used in the test, this is test-specific and assay format specific, and adds cost to the test. This may also not work if the sample contains high titers or levels of anti-streptavidin interference that exceed the test-specific streptavidin interference threshold.

The herein disclosed interference blocking reagents are based on biotin-saturated streptavidin, also called quenched streptavidin (QSAv). The QSAv should be predominantly non-aggregated, that is, based on monomeric streptavidin protein. In some aspects, predominantly non-aggregated QSAv has <5% aggregation by size exclusion chromatography HPLC, of <1% dimer or aggregate, where the average observed molecular weight of the monomer peak is from 52 to 55 KD. In other aspects the QSAv is at least 80, 90, 95, 97, 98, 99% monomeric, or any range bounded by those values. In some embodiments, the streptavidin is blocked, for example with a detergent or polymeric blocking reagents, in order to maintain it in a monomeric, non-aggregated state. QSAv can be used to block anti-streptavidin interference. In some embodiments, the streptavidin may be modified, before, during, or after biotin saturation with one or more additional capture moieties. The capture moiety may be covalently conjugated to the streptavidin before or after the biotin saturation process. Alternatively, the capture moiety may be biotinylated and bound to the streptavidin through biotin-avidin binding before or during the biotin saturation process. However, if the additional capture moiety is biotin—accomplished for example through the use of a bis-biotin linker—it must be bound to the streptavidin in the presence of excess free biotin, that is, during saturation. Embodiments comprising a streptavidin that has been modified with one or more additional capture moieties can be used to block anti-streptavidin interference and interference due to agents that bind the one or more capture moieties.

The herein disclosed interference cleaning reagents are based on streptavidin conjugated to a microparticle (or nanoparticle) to form a streptavidinated bead. The use of a bead, and especially a magnetic bead, facilitates cleaning of a sample or assay reagent without loss or dilution. In some embodiments the streptavidin is saturated with biotin, while in others it is not. Embodiments comprising streptavidin that has been saturated with biotin can be used as a cleaning reagent to remove or reduce anti-streptavidin interference. Embodiments comprising streptavidin that has not been saturated with biotin can be used as a cleaning reagent to remove or reduce both biotin interference and anti-streptavidin interference. In some embodiments, the streptavidin may be modified, before, during, or after biotin saturation with one or more additional capture moieties. The capture moiety may be covalently conjugated to the streptavidin before or after the biotin saturation process. Alternatively, the capture moiety may be biotinylated and bound to the streptavidin through biotin-avidin binding before or during the biotin saturation process. Embodiments comprising a streptavidin that has been modified with one or more additional capture moieties can be used to block anti-streptavidin interference and interference due to agents that bind the one or more capture moieties. The one or more capture moieties can include biotin in those embodiments utilizing a biotin-saturated streptavidin.

The additional capture moieties may be any substance that causes a heterophilic or cross-reactive interference, with the exception that if the streptavidin is not saturated with biotin, the additional capture moiety cannot be biotin. In some embodiments, the additional capture moiety is ruthenium (an element); luminol, acridinium ester, ABEI or cyclic ABEI (like biotin, small organic molecules); or a protein, such as a signal-generating enzyme, for example, alkaline phosphatase or horse-radish peroxidase; streptavidin; an antibody, for example, an antibody from a non-human species; or an antigen. In some embodiments, the antigen is one that can be recognized by antibodies that could cross-react with an antigen being used as a capture moiety of an immunoassay. In various embodiments the antigen used as a capture moiety in the cleaning or blocking reagent, or in the assay, is an allergen, an antigen from a pathogen, or an antigen associated with a disease or disorder such peanut allergens, herpes simplex viral antigens, and autoimmunogens such as cardiac troponin I or TSH with known autoantibody interference issues. In some embodiments the antigen from a pathogen is a viral antigen, a bacterial antigen, a protozoal antigen. In some embodiments, the capture moiety removes cross-reactive antibodies to MERS virus, SARS virus, or other coronaviruses other than SARS-CoV-2. Some embodiments specifically include one or more of these genera or species of capture moiety. Some embodiments specifically exclude one or more of these genera or species of capture moiety.

Biotin linkers, or conjugated biotin, can be constructed or purchased with different linker types and lengths, and different functional groups for covalent attachment of biotin to antibodies, antibody fragments, peptides, oligomers, antigens, and small molecules (conjugated biotin). Common linkers and functional groups (e.g., NHS ester, TFP ester, hydrazide, maleimide, thiol, etc.) used with biotin are NHS-biotin, NHS-LC-biotin, TFP-LC-biotin, NHS-LC-LC-biotin, NHS-chromalink-biotin, NHS-PEO₄-biotin, NHS-(PEO)ₙ-biotin, TFP-(PEO)ₙ-biotin, hydrazide-biotin, hydrazide-LC-biotin, hydrazide-PEO₄-biotin, maleimide-(PEO)ₙ-biotin, and SH-(PEO)ₙ-biotin. Biotin labeling reagents can be amine reactive, carboxyl reactive, carbonyl reactive, water-soluble, and cleavable. Examples include amine reactive, carbonyl reactive, carboxyl reactive, cleavable biotin, click chemistry, desthiobiotin, sulfhydryl reactive, tetrazine ligation, biotin alcohol, bis-biotin-PEG, and D-biotin-PEG-thalidomide.

If a sample contains interference with specificity to biotin, the anti-biotin interference can bind to conjugated biotin used in the test design or assay format and sterically block or impair accessibility of the conjugated biotin to bind to the streptavidin solid phase or other anti-biotin capture moiety. If conjugated biotin can no longer freely bind the anti-biotin capture moiety, just like biotin interference and anti-streptavidin interference, anti-biotin interference will result in a false low assay signal and can result in a false low dose (sandwich assay) or false high dose (competitive inhibition assay).

Tests that use biotin conjugates in the test design, format or formulation cannot simply use biotin or conjugated biotin as a specific blocker, additive or component in assay buffer to mitigate anti-biotin interference in the sample. If used as a blocker in the test, biotin may also compete for and bind to the streptavidin used in the test and result in a false low assay signal and false low dose (sandwich assay) or false high dose (competitive inhibition assay). While low concentrations of biotin below the test-specific biotin interference threshold can be used to block anti-biotin interference, this can be problematic if the patient sample also contains biotin interference close to the interference threshold where the combination or sum of the sample biotin (endogenous biotin) and the test biotin (biotin as a blocker) can exceed the test-specific biotin interference threshold and result in a false low assay signal and false low dose (sandwich assay) or false high dose (competitive inhibition assay).

Streptavidin binds biotin very rapidly and very strongly (with a binding constant reported variously as 10⁻¹⁴ or 10⁻¹⁵ mol/L in the literature). While some studies indicate there is protein structural change or cooperative binding of biotin to the 4 binding sites [28-29], other studies conclude there is no cooperative binding for biotin bound to the four subunits of the tetramer [30-31]. If streptavidin is exposed to a molar excess of free biotin, a very fast and strong binding interaction of biotin to all 4 binding sites will occur and result in 100% biotin saturation (100BS) of all biotin binding sites. Due to having the strongest non-covalent binding interaction known in nature, and very slow off-rate of biotin from streptavidin in normal physiological conditions and pH, 100BS streptavidin will have a very low likelihood of binding additional biotin or conjugated biotin such as biotinylated antibody, protein, oligomers and antigens in a diagnostic test. As used herein, saturation refers to the blocking of biotin binding sites on streptavidin with biotin; this is not biotinylation, the covalent attachment of biotin to streptavidin. Saturated streptavidin can bind anti-streptavidin substances, but will not bind or cross-link biotin bearing substances. Biotin-saturated streptavidin may also be referred to as quenched streptavidin (QSAv).

Streptavidin can be saturated with biotin (i.e. D-biotin) to prepare 100BS streptavidin for use as a blocker to mitigate and manage anti-streptavidin interference. In other embodiments, quenching of streptavidin active biotin binding sites may be accomplished instead by exposing the streptavidin to dissolved biotinylation agents such as Biotin-PEG(n)-COOH or Biotin-PEG(n)-CH3 or Biotin-PEG(n)-OH or other Biotin-R-(non-reactive end chemistry) where R is a carbon chain or ring structure. Saturation involves the exposure of the streptavidin to a molar excess of biotin. In various embodiments, the molar ratio of biotin to streptavidin is in the range of 5:1 to 11:1, or 7:1 to 11:1, or 7:1 to 8:1. In some embodiments, the molar ratio is 7.4:1. In some embodiments, the streptavidin is exposed to all of the saturating biotin making up the stated molar ratio in a single batch. In other embodiments, saturation proceeds through iterative batches, each containing a fraction of the total biotin, with the sum of batches making up the stated molar ratio. For example, one might use three iterative batches in which the biotin:streptavidin ratio is 2:1, instead of a single batch in which it is 6:1; the biotin:streptavidin ratio need not be the same in each of the iterative batches. In some embodiments, a biotin solution and a streptavidin solution are combined by metered addition through a Y-connector. In some embodiments there is an in-line mixer in the tubing connected to the exit of the Y-connector, to ensure rapid, immediate, and complete mixing. A combination of pump speed and tubing length can be used to determine total interaction time in the saturation process. In some embodiments 9 volumes of biotin solution are combined with one volume of streptavidin solution. In some embodiments the streptavidin and biotin solutions are prepared in tris buffered saline, pH 8.5. In one aspect, the starting streptavidin concentration is in the range of 0.1 to 10.0 mg/mL so that the resultant solution of QSAv has a streptavidin concentration in the range of 0.01 to 1.0 mg/mL, but preferably 0.02 to 0.05 mg/mL. Such conditions promote saturation of the biotin binding sites and mitigate non-specific binding of biotin to streptavidin.

In some embodiments, the QSAv is then subjected to a series of hot buffer washes by repeatedly concentrating and re-diluting the QSAv, for example, using diafiltration in a hollow-fiber filter. The wash is used to remove excess and non-specifically bound biotin, so that it does not become a source of interference when the QSAv is used as an interference blocking reagent. In some embodiments, 4-6 or more washes are used, for example, 5 washes, followed by a final concentration step to reduce volume to reach a desired concentration, for example, from 0.1 to 30 mg/mL, or from 1 to 10 mg/mL. In one aspect the volume can be reduced to 5-20% of the original volume of the QSAv solution, for example 10%. In some embodiments, the temperature of the hot wash is 15° C. to 60° C., preferably 40° C. to 55° C., but more preferably 45° C. to 50° C. In some embodiments, the hot wash buffer has a pH in a range of 7.5 to 11, or 8 to 9, for example 8.5. In some embodiments the hot wash buffer has a NaCl concentration in a range of 10 to 500 mM, or 20 to 150 mM, or 25 to 75 mM. In some embodiments the buffer is 10 mM tris, 50-150 mM NaCl. Following washing and the final concentration step, the free biotin concentration should be <1200 pg/mL, for example, <1000, <800, <700, or <600 pg/mL. In some embodiments washed QSAv solution comprises 1-6 pg free biotin/μg streptavidin. In some embodiments, the outflow from the final diafiltration is combined by a metered addition with PBS, appropriately concentrated, to provide QSAv in PBS. In some embodiments for cleaning a sample, a volume of QSAv is added to 400 μl of sample, so that the volume should contain <480 pg of free biotin.

In some embodiments, the outflow from the saturation process is collected for later washing, in other embodiments the outflow from the saturation process feeds directly into the hollow-fiber filter apparatus. The outflow from the saturation process can be divided to feed multiple hollow-fiber filters to increase capacity and avoid excessive back pressure.

These streptavidin solutions are relatively dilute and somewhat prone to aggregation, a problem not encountered with the bead-conjugated streptavidin. This is the reason that the alkaline buffer is used in the saturation and wash procedures for producing the QSAv. (By contrast, water is used in the analogous washing of the bead-conjugated streptavidin.) Aggregation can be further mitigated by including 0.01-1% w/v TWEEN 20 or another surfactant in the wash buffer. Aggregation can be further mitigated by covalent modification with a preparative blocking reagent, for example, by PEGylation of the streptavidin, prior to saturating with biotin. Thus, in some embodiments, the QSAv is a blocked, monomeric, biotin-saturated streptavidin. In some embodiments, monomeric QSAv is <5% aggregates by size-exclusion chromatography HPLC; in other embodiments the monomeric QSAv is <1% aggregates.

In one embodiment, 100BS streptavidin (QSAv) can be used as a blocking reagent or protein blocker to target and deplete anti-streptavidin interference in a sample. 100BS streptavidin can be added to an assay buffer, blocking buffer or test components used in the test formulation such as the detection antibody, or any combination thereof, to mitigate the susceptibility of the test to anti-streptavidin interference. In another embodiment, streptavidin is covalently conjugated to a microparticulate binding surface and subsequently incubated with a molar excess of biotin to prepare 100BS streptavidin-beads. The 100BS streptavidin-beads can be used to pre-treat a sample to target and deplete anti-biotin interference prior to the diagnostic test.

The herein disclosed interference cleaning reagents are based on streptavidin-conjugated beads. In some embodiments the streptavidin is quenched (saturated) with biotin after it is attached to the core particle. In some embodiments the streptavidin is quenched with biotin before it is attached to the core particle, for example, using the QSAv described herein. In some embodiments, the core particle is magnetic. In some embodiments, the core particle is 500 nm, or about half a μm, in diameter and can therefore be referred to as either a nanoparticle or a microparticle. In some embodiments the core particle is covalently conjugated to streptavidin using 1-Ethyl-3-(3-dimethylaminopropyl)carbodiimide chemistry. To remove passively adsorbed streptavidin, and to prevent non-specific binding to the bead in further preparative steps and when in use as an interference cleaning reagent, the bead surface is conditioned with stripping reagents (salts, detergents, low and high pH) and the beads blocked using detergents and polymeric blocking reagents. This also promotes nanoparticle monodispersion and colloidal stability. To produce 100BS streptavidin beads, biotin quenching of the streptavidin can be carried out analogously to the production of 10BS streptavidin (QSAv) above. Saturation involves the exposure of the bead-conjugated streptavidin to a molar excess of biotin. As above, saturation of streptavidin active biotin binding site may be accomplished instead by exposing the streptavidin to dissolved biotinylation agents such as Biotin-Peg(n)-COOH or Biotin-Peg(n)-CH3 or Biotin-Peg(n)-OH or other Biotin-R-(non-reactive end chemistry) where R is a carbon chain or ring structure In various embodiments, the molar ratio of biotin to streptavidin is in the range of 4:1 to 6:1, for example 5:1. The effective molar excess of biotin is somewhat higher than this formal ratio, as some of the biotin binding sites will be inaccessible due to steric hindrance by the core particle. In some embodiments, the streptavidinated beads are suspended, and the biotin solution made up in, PBS, pH 6.8. In some embodiments, the biotin solution and bead suspension are combined in a vessel and mixed, for example, for 1 hour at room temperature. In some embodiments, the streptavidinated bead suspension and the biotin solution are combined by metered addition, essentially as described above in the production of QSAv.

In some embodiments, the biotin-saturated streptavidinated beads are subjected to a hot wash to remove passively adsorbed biotin, so that it does not leach off in use and become a source of interference. Unlike the 100BS streptavidin above, washing the 100BS streptavidin beads can make use of filtration, sedimentation, or magnetic separation, as an alternative to diafiltration. The wash can also differ from the 100BS streptavidin wash in using hot alkaline (pH 7.5) water instead of buffer. The wash suspensions are also sonicated. In some embodiments, the suspension of washed 100BS streptavidin beads has a free biotin concentration of <1200 pg/mL, for example <1000, <800, <600, <400, or <200 pg/mL. In some embodiments, the suspension of washed 100BS streptavidin beads comprises 5-30 pg free biotin/pg streptavidin. In some embodiments for cleaning a sample, a volume of 100BS streptavidin beads is added to 400 μl of sample, so that the volume should contain <480 pg of free biotin.

Free biotin (i.e. D-biotin) can also be added to the 100BS streptavidin or 100BS streptavidin-beads storage solution, assay buffer or test components to improve stability of 100BS streptavidin or 100BS streptavidin-beads and to ensure the streptavidin remains 100% saturated with biotin over time. In one embodiment, 100BS streptavidin in a storage solution, assay buffer, or test component containing excess biotin can be used as a blocking reagent to target both anti-streptavidin and anti-biotin interference mechanisms with a single blocking reagent. In one embodiment, the blocking reagent can be used prior to a test to pre-treat a sample, thereby blocking anti-streptavidin and/or anti-biotin interference mechanisms. In one embodiment, the blocking reagent can be used within a diagnostic test or assay design, such as in an assay buffer or reagent buffer, to block and mitigate interference mechanisms during the test. In one embodiment, free biotin can be added to 100BS streptavidin or 100BS streptavidin-beads at concentrations within the physiological range up to 1,100 pg/mL. In another embodiment, free biotin can be added to 100BS streptavidin or 100BS streptavidin-beads at high concentrations such as 100,000 pg/mL (100 ng/mL) or 1,000,000 pg/mL (1,000 ng/mL). If 100BS streptavidin or 100BS streptavidin-beads are stored in a solution containing free biotin, it will remain 100BS streptavidin since any biotin that dissociates from streptavidin will be immediately replaced with another biotin from the biotin added to the storage solution due to the very strong binding constant and fast on-rate of streptavidin to biotin. Therefore, 100BS streptavidin, or biotin quenched streptavidin, can be used as a blocking reagent for streptavidin-based tests or immunoassays where 100BS streptavidin is added to the assay buffer which also contains sub-physiological biotin concentrations for stability. If any biotin dissociates from the streptavidin blocker it will be replaced with the biotin added to the assay buffer, and the amount of biotin subsequently added to the sample or test reaction from this assay buffer would be minimal and within the physiological biotin concentration range.

IVD companies test for and provide test-specific biotin interference thresholds in their package inserts (PI) or instructions for use (IFU) for each assay susceptible to biotin interference [9, 14-15]. Tests with biotin interference thresholds <51 ng/mL, such as the Ortho Clinical Diagnostics Vitros cardiac TnI test with a threshold of 2.4 ng/mL, are considered high risk tests, or vulnerable immunometric and competitive methods [9]. While biotin can be added to the 100BS streptavidin or 100BS streptavidin-beads storage solution to improve stability and ensure 100% saturation over time, the final free biotin concentration must be less than the test-specific biotin interference threshold to mitigate test interference from the biotin added to the storage solution. In one embodiment, 100BS streptavidin or 100BS streptavidin-beads is stored in a biotin solution with biotin concentrations within the physiological range, or <1,100 pg/mL, such that it will not interfere in the test. In another embodiment, 100BS streptavidin-beads are stored is a biotin solution containing biotin >1,100 pg/mL such as 2, 5, 10, 20, 30, 50, 100, 250 or 500 ng/mL biotin. The 100BS streptavidin-beads are subsequently filtered, centrifuged, or magnetically separated from the sample, or combinations thereof, to remove the biotin storage solution from the 100BS streptavidin-beads prior to the addition of the sample to the 100BS streptavidin-beads. Removal of the biotin storage solution immediately prior to 100BS streptavidin-bead use will decrease free biotin concentrations to less than 1,100 pg/mL, to less than 500 pg/mL, or preferably to less than 100 pg/mL, and ensure the free biotin concentration is below the biotin interference threshold of the test.

The primary amines (R—NH₂) of 100BS streptavidin and 100BS streptavidin-beads can be covalently conjugated to biotin using amine reactive biotin labeling reagents such as NHS-biotin, NHS-LC-biotin, NHS-LC-LC-biotin, NHS-chromalink-biotin, NHS-PEO₄-biotin, and NHS-(PEO)ₙ-biotin, TFP-(PEO)ₙ-biotin (amine reactive means) and by performing the biotin conjugation in a molar excess of free biotin to mitigate the binding and capture of the biotin labeling reagent by the streptavidin biotin binding sites. In one embodiment, streptavidin is covalently conjugated to a microparticulate binding surface, the microparticulate binding surface is conditioned (blocked and stripped) such that there only remains covalently attached streptavidin, the streptavidin conjugated microparticulate binding surface is exposed to a molar excess of free biotin (D-biotin) to prepare 100BS streptavidin-beads, a molar excess of free biotin is added to the 100BS streptavidin-bead storage solution such as PBS pH 7.4, and the primary amines of the 100BS streptavidin is conjugated to NHS-PEO₄-biotin to prepare biotinylated 100BS streptavidin-beads. Biotinylation of streptavidin conjugated beads refers to the covalent conjugation of biotin to the magnetic particle (or streptavidin thereon) and should not be confused or equated with saturation of the biotin-binding sites of streptavidin. Biotinylated streptavidin beads can bind anti-biotin substances (in addition to anti-streptavidin substances). The biotin used to saturate the streptavidin generally will not bind the most problematic anti-biotin substances, as the necessary portions of the biotin molecule are engaged with the streptavidin. In another embodiment, 100BS streptavidin or 100BS streptavidin beads have thiol groups (sulfhydryl groups, or R—SH) introduced on the streptavidin via thiolation of the streptavidin primary amines (R—NH₂) using standard thiolation chemistry known in the art such as Succinimidyl trans-4-(maleimidylmethyl)cyclohexane-1-Carboxylate (SMCC), Succinimidyl 3-(2-Pyridyldithio)Propionate (SPDP), SPDP-PEG_{(4,6,8,12,24, or 136)}-NHS ester, SPDP NHS ester, SPDP-C6-NHS ester, SPDP-C6-Sulfo-NHS ester, PC SPDP-NHS carbonate ester, and SPDP-C6-Gly-Leu-NHS ester (means for thiolation). If SPDP is used, the SPDP conjugated to the streptavidin is cleaved using TCEP and EDTA, and the SPDP leaving group is washed, desalted or dialyzed away leaving only SH—R conjugated 100BS streptavidin. After preparing 100BS thiolated streptavidin with a molar excess of biotin, the thiols (R—SH) of 100BS streptavidin and 100BS streptavidin-beads can be covalently conjugated to biotin using a thiol reactive or sulfhydryl reactive biotin labeling reagents such as maleimide-PEO_{(2,3,6, or 11)}-biotin and Biotin-SPDP (thiol or sulfhydryl reactive means). The sulfhydryl reactive biotin labeling is performed in a PBS pH 6.8 buffer containing EDTA (up to 2 mM), TCEP (<1 mM), and a molar excess of free biotin to 1) reduce the thiols and mitigate disulfide bonds or bridging, and 2) to mitigate the binding and capture of the biotin labeling reagent by the streptavidin biotin binding sites. Since maleimide groups are 1000-fold more reactive toward free sulfhydryls than amines at pH 6.5 to 7.5, and at pH>8.5 maleimide groups favors primary amines, the maleimide conjugation is carried out at pH 6.8 for minimizing the reaction toward primary amines. As an alternative to SPDP, analogous reagents based on N-succinimidyl-S-acetyl-thioacetate (SATA) can be used.

In another embodiment, 100BS streptavidin or 100BS streptavidin beads have maleimide groups introduced on the streptavidin primary amines (R—NH₂) using standard ester-maleimide heterobifunctional crosslinking chemistry known in the art such as succinimidyl 4-(N-maleimidomethyl)cyclohexane-1-carboxylate (SMCC), maleimide-PEG-NHS ester, maleimide-PEO_{(1,2,3,4,5,6,8 or 12)}-NHS ester, or maleimide-PEG_{(1,2,3,4,5, or 6)}-PFP (means for maleimidation). After preparing 100BS maleimide streptavidin with a molar excess of D-biotin, the maleimides of 100BS streptavidin and 100BS streptavidin-beads can be covalently conjugated to biotin using a maleimide reactive biotin labeling reagents such as biotin-PEG-SH or biotin-PEG-thiol, where the PEGₙ or PEOₙ can be different lengths such as n=1, 2, 3, 4, 5, 6, 8 or 12. The maleimide reactive biotin labeling is performed in a PBS pH 6.8 buffer containing EDTA (up to 2 mM), TCEP (<1 mM), and a molar excess of free biotin to 1) reduce the thiols and mitigate disulfide bonds or bridging of the biotin labeling reagent, and 2) to mitigate the binding and capture of the biotin labeling reagent by the streptavidin biotin binding sites. Since maleimide groups are 1000-fold more reactive toward free sulfhydryls than amines at pH 6.5 to 7.5, and at pH>8.5 maleimide groups favors primary amines, the maleimide biotin conjugation is carried out at pH 6.8.

Similar ester, thiol, or maleimide chemistry is also applicable to embodiments in which the streptavidin is not biotin-saturated. For example, a ruthenium ester can be reacted with the primary amines of the streptavidin.

In a specific embodiment, 1) streptavidin is covalently conjugated to a microparticulate binding surface, 2) the microparticulate binding surface is conditioned such that there only remains covalently attached streptavidin on the microparticulate binding surface and the surface has very low non-specific binding, 3) the streptavidin conjugated microparticulate binding surface is exposed to a molar excess of free biotin (D-biotin) to prepare 100BS streptavidin-beads, 4) 100BS streptavidin-beads are covalently conjugated to biotin using a biotin labeling reagent while in the presence of a molar excess of free biotin 5) biotinylated 100BS streptavidin-beads are filtered, centrifuged or magnetically separated to remove the buffer and excess biotin, 6) the biotinylated 100BS streptavidin-beads are washed with multiple cycles of 50° C. water and resuspended in a storage solution to arrive at the finished reagent.

In a specific embodiment, 1) biotinylated 100BS streptavidin-beads are filtered, centrifuged, or magnetically separated to remove the storage solution, 2) a sample containing anti-streptavidin interference, anti-biotin interference, or both interferences is added to the biotinylated 100BS streptavidin-beads to pre-treat the sample, 7) sample interference is depleted or decreased below the assay blocking threshold (ABT) or test interference threshold, 8) the biotinylated 100BS streptavidin-beads are filtered, centrifuged or magnetically separated from the sample, and 9) the essentially bead-free sample supernatant is aspirated and tested by the diagnostic test to report an accurate test result.

In a specific embodiment, 100BS streptavidin-beads are used to pretreat a sample to bind anti-streptavidin interference and deplete anti-streptavidin interference below the assay blocking threshold (ABT), or below the test interference threshold, prior to the diagnostic test. In another embodiment, biotinylated 100BS streptavidin-beads are used to pretreat a sample to bind anti-biotin interference and deplete the anti-biotin interference below the assay blocking threshold (ABT), or below the test interference threshold, prior to the diagnostic test. In another embodiment, biotinylated 100BS streptavidin-beads are used to pretreat a sample to bind both anti-streptavidin and anti-biotin interference from the same sample simultaneously and deplete both interferences below the assay blocking threshold (ABT) or test interference threshold priorto the diagnostic test.

There are currently no fast and easy-to-use product solutions available to detect, characterize and mitigate biotin, anti-biotin and anti-streptavidin interference in patient samples. In a specific embodiment, streptavidin-beads (Bead 1), 100BS streptavidin-beads (Bead 2), and biotinylated 100BS streptavidin-beads (Bead 3) can be used systematically or sequentially to detect and determine which interference mechanism or mechanisms are present in the sample. The sample with suspect interference is tested neat (no beads added) and is the control result. Three different aliquots of the sample are treated with Bead 1 (aliquot 1), Bead 2 (aliquot 2) and Bead 3 (aliquot 3), respectively. The three pre-treated aliquots are re-tested and the test result for each Bead type are compared to the control test result (Table 1). If the test result of the control is similar to the test results from Bead 1, 2 and 3 pre-treatments, sample interference is unlikely and can be ruled-out. However, if the Bead 1 pre-treatment result is significantly different than the control result, biotin interference and/or anti-streptavidin interference are likely, and sample interference can be ruled-in. If the Bead 2 pre-treatment result is significantly different than the control result, anti-streptavidin interference is likely, and sample interference can be ruled-in. If the Bead 3 pre-treatment result is significantly different than the control result, anti-streptavidin interference and/or anti-biotin interference are likely, and sample interference can be ruled-in. If the Bead 1 pre-treatment result is significantly different than the control result, but the Bead 2 and Bead 3 pre-treatment results are similar to the control, biotin interference can be ruled-in. If the Bead 1 and Bead 2 pre-treatment results are similar to the control result, but the Bead 3 result is significantly different than the control result, anti-biotin interference can be ruled-in. If Bead 1, Bead 2 and Bead 3 pre-treatment results are all significantly different than the control result, anti-streptavidin interference can be ruled-in.

TABLE 1

| | Result | Result | Result | Result | |
|---|---|---|---|---|---|
| Pre-Treatment | 1 | 2 | 3 | 4 | Result5 |
| Bead 1 | − | + | − | + | + |
| (streptavidin-beads) | | | | | |
| Bead 2 (100BS | − | − | − | + | + |
| streptavidin beads) | | | | | |
| Bead 3 (biotinylated | − | − | + | − | + |
| 100BS streptavidin | | | | | |
| beads) | | | | | |
| Biotin Interference | No | Yes | No | Yes | No |
| Anti-streptavidin | No | No | No | Yes | Yes |
| Interference | | | | | |
| Anti-Biotin | No | No | Yes | No | No |
| Interference | | | | | |
| Interference Likely | Rule-Out | Rule-In | | | |

Possible outcomes by using 3 different sample pre-treatment reagents Bead 1, Bead 2 and Bead 3, and comparing results against the control; similar (−) and different (+). It is not likely for Bead 1+, Bead 2−, and Bead 3+, as anti-biotin interference would bind free biotin unless it only recognizes conjugated biotin. It is not possible for Bead 1−, Bead 2+, and Bead 3−, as anti-streptavidin interference would be depleted by both Bead 1 and Bead 2. As shown in Result 4, Bead 3 may not deplete anti-streptavidin interference (−) if conjugated biotin sterically blocks or interferes with anti-streptavidin antibody or protein binding.

Producing a free (or soluble) biotin-saturated streptavidin (quenched streptavidin; QSAv) is largely analogous to producing the biotin-saturated, streptavidin conjugated beads. Moreover, the streptavidin can be conjugated to additional moieties to serve as capture moieties or to block sites that could promote aggregation of the streptavidin. Such conjugation can be carried out before or after quenching (unless the additional capture moiety is to be biotin, in which case it can only be carried out after quenching). A somewhat higher molar ratio of biotin to streptavidin, of about 7 to 8, is used than the 5:1 ratio used in the minimal saturation procedure for the beads. This is because some of the biotin binding sites on the bead-conjugated streptavidin will be sterically hindered, so that the effective ratio is somewhat higher than the formal ratio.

The QSAv is preferably predominantly monomeric. In various embodiments the QSAv is at least 80, 90, 95, 97, 98, 99% monomeric, or any range bounded by those values. To ensure that the reagent is, and remains, monomeric and does not form aggregates, the streptavidin can be blocked with detergents or polymeric blocking reagents. Blocking could include PEGylation. There is a large variety of commercially-available PEGylation reagents of various size and chemical modification such as from ThermoFisher Scientific, Broadpharm, Quanta Biodesign and Creative Pegworks. One example would be NHS-ester-PEG(4)-OH. Other examples include TFP-(PEO)n-OH or TFP-(PEG)n-OH (Quanta Biodesign). These can be covalently attached to any exposed lysine residues on the streptavidin via NHS-ester chemistry. Alternatively, TFP-(PEG)n-COOH or NHS-(PEG)n-COOH could be attached to lysine residues through EDC chemistry. Many other alternatives will be familiar to the person of skill in the art. Preparation of monomeric QSAv can also be accomplished by biotin quenching within a buffer containing kosmotropic reagents such as urea, imidazole, trehalose or others.

### EXAMPLES

The following non-limiting examples are provided for illustrative purposes only in order to facilitate a more complete understanding of representative embodiments now contemplated. These examples should not be construed to limit any of the embodiments described in the present specification.

### Example 1

### Method to Prepare Biotinylated Streptavidin Coated Magnetic Nanoparticles or Biotinylated 100BS Streptavidin-Beads

550-600 nm magnetic carboxylic acid nanoparticles were covalently conjugated to streptavidin using EDC (1-Ethyl-3-(3-dimethylaminopropyl)carbodiimide) chemistry, the bead surface conditioned with stripping reagents (salts, detergents, low and high pH) to remove passively absorbed streptavidin, and the beads blocked using detergents and polymeric blocking reagents to decrease non-specific binding and promote nanoparticle monodispersion and colloidal stability. The total concentration of streptavidin covalently conjugated to the beads was determined to be 17.69 micrograms per milligram (μg/mg) using a modified micro BCA total protein assay. The final bead concentration was determined gravimetrically and was adjusted to 10.0 milligrams beads per milliliter (mg/mL) in PBS, 2 mM EDTA, pH 6.8.

A 10.0 mg/mL stock solution of D-biotin (Sigma, Part Number B4601-100MG, Lot SLBS8478, MW 244.31) in PBS, pH 7.4 was by prepared by making a 100 mg/mL concentrated stock solution of D-biotin in DMSO (Baker, Part Number 9224-01, Lot 0000217025), or 10 mg D-biotin was added to 100 μL DMSO and mixed. Once the D-biotin was completely and homogenously dissolved in the DMSO, 900 μL of PBS, pH 7.4 was added and mixed to prepare a 1.0 mL 90:10 (PBS:DMSO) stock solution of D-biotin at 10.0 mg/mL.

A total of 2.5 mL of the streptavidin magnetic nanoparticles was aliquoted and dispensed into a reaction vial which corresponded to a total of 442.25 μg streptavidin: [(25 mg beads)×(17.69 μg streptavidin/mg beads)]. A total of 442.25 μg streptavidin corresponds to 0.00804 μM streptavidin: [(442.25 μg streptavidin)/(55,000 μg streptavidin/μM streptavidin).

A 1000-fold molar excess of D-biotin over total moles streptavidin was prepared by adding 1,964.475 μg D-biotin to 25 mg of streptavidin conjugated magnetic particles at 17.69 μg streptavidin/mg beads, or to 442.25 μg streptavidin: [(0.00804 μM×1000)×(244.31 μg biotin/μM)]. To add a 1000-fold molar excess of D-biotin to 0.00804 μmol streptavidin, 200 μL of the 10.0 mg/mL D-biotin stock solution, or 2,000 μg D-biotin, was added to 25 mg of streptavidin magnetic nanoparticles at 17.69 μg streptavidin/mg beads in PBS, 2 mM EDTA, pH 6.8. The streptavidin magnetic nanoparticles were incubated with the D-biotin with mixing for 1 hour at room temperature to saturate the streptavidin biotin binding sites 100% with biotin and prepare the 100BS streptavidin-beads.

To covalently conjugate the 100BS streptavidin-beads to biotin in a molar excess of D-biotin, a 100-fold molar excess of NHS-PEG₄-biotin (Broadpharm, Part Number 20566, Lot B93-039, MW 588.7), or 500 μg NHS-PEG₄-biotin, was added to 25 mg of streptavidin conjugated magnetic particles at 17.69 μg streptavidin/mg beads, or 442.25 μg streptavidin, or 0.00804 μM streptavidin. 5 mg NHS-PEG₄-biotin was added to 100 μL DMSO and mixed to prepare a 50.0 mg/mL stock solution of NHS-PEG₄-biotin in DMSO. A 100-fold molar excess of NHS-PEG₄-biotin, or 473.315 μg NHS-PEG₄-biotin [(0.804 μM NHS-PEG₄-biotin)×100)×(588.7 μg biotin/μM)] was added to 100BS streptavidin-beads by adding 10.0 μL of a 50.0 mg/mL stock solution of NHS-PEG₄-biotin in DMSO to 25 mg of 100BS streptavidin-beads at 17.69 μg streptavidin/mg beads in PBS, 2 mM EDTA, pH 6.8 with 1000-fold molar excess of D-biotin from the saturation step and mixed for 1 hour at room temperature. The biotinylated 100BS streptavidin-beads were washed 4 times with PBS, pH 7.4 to wash away the excess NHS-PEG₄-biotin.

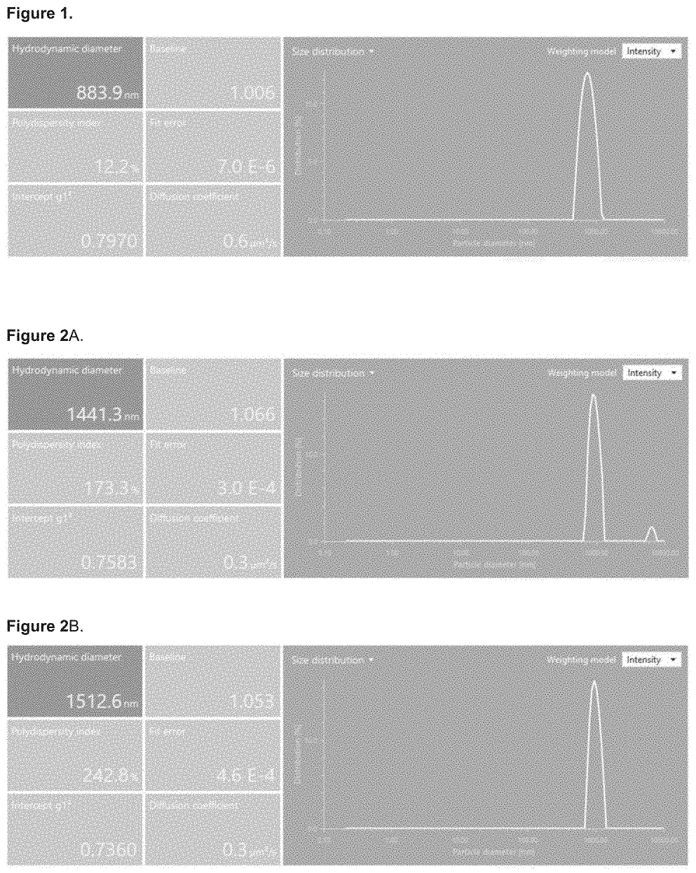

To verify that the 100BS streptavidin-beads were successfully conjugated to biotin without any bead aggregation from streptavidin mediated binding of conjugated biotin on a different bead (i.e. crosslinking of beads), the biotinylated 100BS streptavidin beads were analyzed by particle size measurement using an Anton Paar Litesizer 500 analyzer. The mean size distribution was 883.9 nm (FIG. 1).

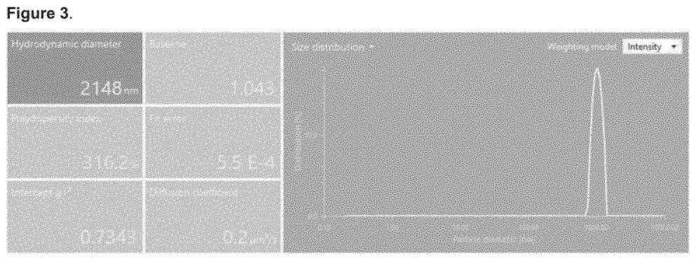

To demonstrate biotin was successfully conjugated to the streptavidin on the 100BS streptavidin-beads, a limiting amount of native streptavidin was added to the biotinylated 100BS streptavidin-beads to promote bead aggregation from streptavidin mediated crosslinking of the beads. A total of 50 μg streptavidin was added to 25 mg of biotinylated 100BS streptavidin-beads and incubated for 4 hours at room temperature. The beads demonstrated aggregation 30 minutes after streptavidin addition with peaks at 1,441.3 nm and 6,641 nm and polydispersity index of 173.3% (FIG. 2A), and peaks at 1,512.6 nm and 14,536 nm with a polydispersity index of 242.8% (FIG. 2B).

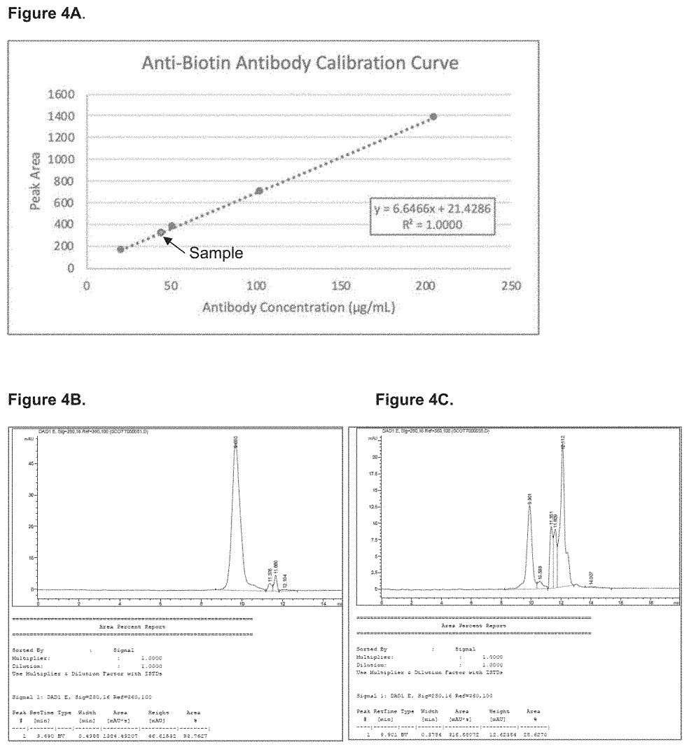

A similar bead aggregation was performed using an anti-biotin conjugate monoclonal antibody that recognizes conjugated biotin with a similar affinity as streptavidin, but recognizes free biotin at a million times lower affinity than streptavidin, or an antibody which specifically binds to biotin of a biotin conjugate and which has a higher affinity for biotin of a biotin conjugate than for free biotin (WO2020/028776; VeraBind Biotin™, Veravas). The antibody was added to the biotinylated 100BS streptavidin-beads and incubated overnight at room temperature. The beads demonstrated aggregation with a peak of 2,148 nm and polydispersity index of 316.2% (FIG. 3).

### Example 2

### Method to Use Biotinylated Streptavidin Coated Magnetic Nanoparticles, or Biotinylated 100BS Streptavidin-Beads, to Deplete Anti-Biotin Antibody from Sample

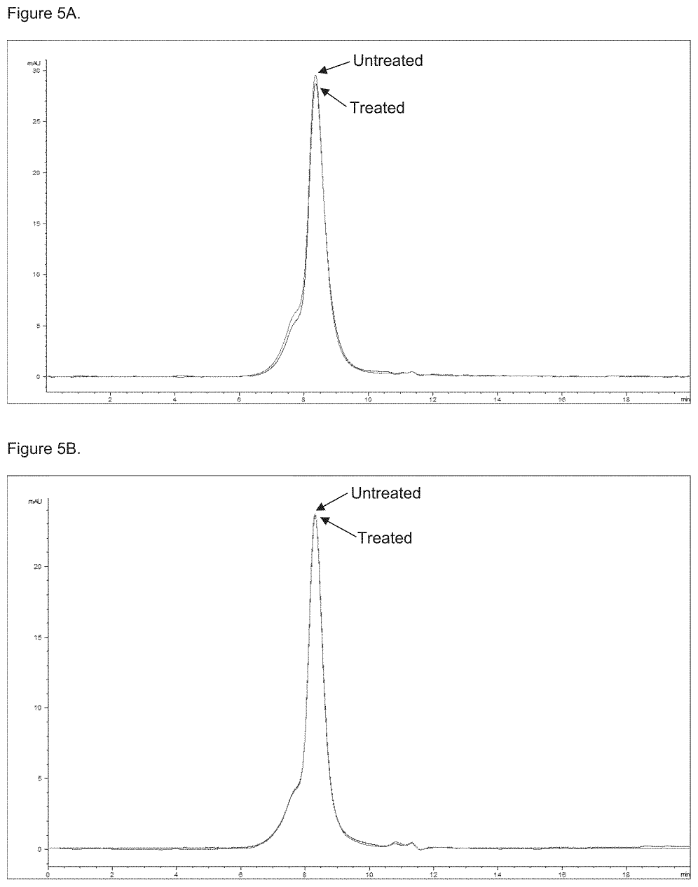

To demonstrate successful biotinylation of the 100% biotin saturated streptavidin conjugated magnetic nanoparticles, or 100BS streptavidin-beads, lyophilized mouse ascites containing a monoclonal anti-biotin antibody with specificity for conjugated biotin was purified using Melon Gel purification Kit (ThermoFisher, Part Number 45214) and ascites conditioning buffer (ThermoFisher, Part Number 45219, Lot TB263120), and desalted in PBS pH 7.2 using Zeba Spin Desalting Columns, 40K MWCO (ThermoFisher, Part Number 87770). The final concentration of the anti-biotin antibody was 0.205 mg/mL, and 50 μL of the anti-biotin antibody, or 10.25 μg anti-biotin antibody, was added to 950 μL of PBS, pH 7.4 in a glass HPLV vial to make a 10.25 μg/mL anti-biotin stock solution. 100 μL, 50 μL, 25 μL and 10 μL of the anti-biotin stock solution was sequentially injected onto a Phenomenex s4000 SEC HPLC column, flow rate of 1.0 mL/min, and mobile phase PBS pH 7.4, and Peak Area was determined for each concentration of antibody injected to generate a calibration curve of Peak Area (Y-axis) vs. Antibody Concentration (X-axis): y=6.6466×+21.4286, R²=1.0000 (FIG. 4A).

Next, 750 μL of the anti-biotin antibody at 0.205 mg/mL was pre-treated with the biotinylated 100BS streptavidin-beads by:
1. Remove the biotinylated 100BS streptavidin-beads Reagent vial from storage and vortex for a minimum of 10 seconds at medium speed.
2. Place an empty 2 mL Sarstedt Micro tube into the VeraMag 400™ magnetic separator until the collar of the tube contacts the magnet frame.
3. Dispense 750 μL, or 7.5 mg beads at 10.0 mg/mL, of the well-mixed reagent into a 2 mL Sarstedt Micro tube.
4. Wait at least 30 seconds, carefully aspirate and discard all of the supernatant without disturbing the pellet of magnetic nanoparticles.
5. Dispense 750 μL of well-mixed anti-biotin antibody at 0.205 mg/mL.
6. Cap the tube and vortex the sample for a minimum of 10 seconds at medium speed.
7. Place the tube onto a rotating mixer at medium speed and incubate at RT for 30 minutes.
8. Loosen the screw cap and place the tube into the VeraMag 400 until the collar of the tube contacts the magnet frame.
9. Allow the nanoparticles to magnetically separate from the sample for 5 minutes.
10. Carefully aspirate the sample, without disturbing the pellet of magnetic nanoparticles, and dispense into a clean tube. All of the sample can be aspirated if this step is performed carefully. NOTE: If any of the magnetic nanoparticles are accidentally aspirated then simply return the mixture to the tube, cap the tube, and return to step 9.
11. The conditioned sample is now ready for analysis.
12. Filter the sample using a 0.2 micron cellulose-acetate syringe filter. The average protein loss using this filter was 16.5 μg).
13. Inject 100 μL of the sample on the Phenomenex s4000 SEC HPLC column, flow rate of 1.0 mL/min, and mobile phase (50 mM potassium phosphate, 250 mM potassium chloride, pH 6.8), and determine the Peak Area at Retention Time ˜9.6 to 9.9 minutes.
14. Based on the calibration curve equation, solve for x (μg/mL antibody) by using the Peak Area of the antibody peak (y).

The Peak Area of 100 μL of the anti-biotin Ab sample was 1,384 (FIG. 4B), and the concentration this Peak Area corresponds to on the calibration curve is 205 μg/mL (FIG. 4A). The Peak Area of 100 μL of the pre-treated and depleted anti-biotin sample was 318 (FIG. 4C), and the concentration this Peak Area corresponds on the calibration curve was 44.62 μg/mL (FIG. 4A). Since the starting volume of the antibody pre-treated with the depletion reagent was 750 μL, this corresponds to 33.465 μg antibody: [44.62 μg/mL×0.750 mL]. Since 16.5 μg antibody was lost to the 0.2 micron cellulose-acetate syringe filter, the total remaining antibody after sample pre-treatment was 49.965 μg antibody: [33.465 μg+16.5 μg]. The starting quantity of anti-biotin antibody pre-treated was 153.75 μg antibody: [205 μg/mL×0.750 mL]. The % anti-biotin antibody captured and depleted by the biotinylated 100BS streptavidin-beads was 67.5%: [((153.75 μg-49.965 μg)/153.75 μg)×100%].

This study demonstrated the biotinylated 100BS streptavidin-beads were able to deplete 103.785 μg antibody: (153.75 μg-49.965 μg). This corresponds to a binding capacity of 13.838 μg of anti-biotin antibody per mg biotinylated 100BS streptavidin-beads: [(103.785 μg antibody)/(7.5 mg beads)].

### Example 3

### Preparation of Biotinylated 100BS Streptavidin-Beads Using a Low Molar Excess of Biotin

Use of a 1000-fold molar excess of free biotin for biotin saturation of streptavidin coated beads can lead to non-specific association of biotin with streptavidin or the bead. In use, while successfully depleting anti-biotins and anti-streptavidins, the non-specifically associated biotin can leach off of or dissociate from of the 100BS streptavidin-beads and potentially cause biotin interference in the assay. Several approaches to remove or mitigate this non-specific biotin binding were investigated, including saturating the streptavidin prior to conjugation to the magnetic nanoparticle, various washing procedures after saturation; use of lower molar excesses of biotin; and various biotin-linker molar excesses. Ultimately a combination of low molar excess of biotin and particular washing conditions produced a product without a free biotin leaching problem.

After conjugation of the streptavidin to the magnetic nanoparticle, but prior to biotinylation, streptavidin beads were exposed to a 5-fold molar excess of free biotin (that is, a 5:1 mole ratio of biotin:streptavidin or 5:4 ratio of biotin:biotin binding sites). The saturated bead was then washed with water at 50° C. with sonication. (The effective ratio of biotin to biotin binding sites is somewhat higher as conjugation of the streptavidin to the bead leads to steric hindrance of some of the biotin binding sites.

Biotinylation was carried out with 4-, 25-, and 50-fold molar excesses of the biotinylation reagent (biotin-PEG₄-NHS linker). It was found that the 50-fold molar excess gave optimal results.

The finished beads were tested for neutrality to demonstrate that the beads did not themselves cause an interference when used to pre-treat a serum sample according to the following protocol:
1. Remove the Biotinylated 100BS streptavidin-beads reagent vial from storage and vortex for a minimum of 10 seconds at medium speed to mix well and resuspend the reagent.
2. Insert the reagent vial in the foam vial holder.
3. Insert an empty Micro tube 2 ml (SARSTEDT Order Number 72.694) into a VeraMag™ magnet (Veravas) until the collar of the tube contacts the magnet frame.
4. Dispense 200 μL of the well-mixed reagent (beads) into the empty tube to separate the reagent on the magnet for >30 seconds to form a reagent pellet.
5. Carefully aspirate and discard all of the storage buffer supernatant (˜200 μL) without disturbing the reagent pellet.
6. Dispense 400 μL of well-mixed serum or plasma sample into the tube containing the reagent pellet.
7. Tighten the screw cap on the tube, remove the tube from the magnet, and vortex for a minimum of 10 seconds at medium speed to mix well and resuspend the reagent in the sample.
8. Place the tube onto a laboratory mixer at medium speed and incubate at room temperature for 10 minutes.
9. Loosen and remove the screw cap and insert the tube into the magnet until the collar of the tube contacts the magnet frame.
10. Magnetically separate the reagent for >4 minutes to form a reagent pellet.
11. Carefully aspirate the sample supernatant without disturbing the reagent pellet and dispense the sample into a transfer tube for testing. Note: All of the sample supernatant (˜400 μL) can be aspirated if this step is performed carefully. If any of the reagent is accidentally aspirated then simply return the sample/reagent mixture to the tube and return to step 10.
12. The sample is now ready for testing.

Pre-treated samples were then used in the Roche Elecsys TSH assay, as an example of a sandwich immunoassay, and the Roche Elecsys FT4 assay, as an example of a competitive immunoassay (see Table 2). There was no significant analytical or clinical difference in results for Treated vs. Untreated for all sample tested by both assays.

TABLE 2

| | Analyte | | | | | | |
|---|---|---|---|---|---|---|---|
| | Concen- | Un- | | | | Differ- | |
| Immunoassay | tration | treated | | Treated | | ence | |
| Sandwich | Low | 0.2 | mIU/L | 0.1 | mIU/L | −0.1 | mIU/L |
| Sandwich | Medium | 2.6 | mIU/L | 2.5 | mIU/L | −0.1 | mIU/L |
| Sandwich | High | 5.9 | mIU/L | 6.0 | mIU/L | 0.1 | mIU/L |
| Competitive | Low | 0.7 | ng/dL | 0.7 | ng/dL | 0.0 | ng/dL |
| Competitive | Medium | 1.2 | ng/dL | 1.2 | ng/dL | 0.0 | ng/dL |
| Competitive | High | 1.9 | ng/dL | 1.9 | ng/dL | 0.0 | ng/dL |

### Example 4

### Preparation of Validation Lots

Three lots of beads were prepared essentially as described in Example 3, that is, with a 5:1 molar ratio of biotin to streptavidin for the saturation, a 50-fold molar excess of biotin-PEG₄-NHS linker, and 50° C. washes with sonication, using different sources of streptavidin. One lot, FSAv, used fresh streptavidin; one lot, RSAv, used streptavidin reclaimed from previous bead coating reactions; and one lot, MSAv, used a mixture of 80% reclaimed streptavidin and 20% fresh streptavidin. The streptavidin content of the three lots was 35 μg/mg beads for lot FSAv, 30 μg/mg beads for RSAv, and 19 μg/mg beads for MSAv. The three lots were then used in the validation studies described in Examples 5-8, below.

The conjugation of streptavidin to the magnetic nanoparticle uses an excess of streptavidin. Rather than discarding the unconsumed reagent, it can be recovered by filtering, desalting, and concentration. It has be found that such reclaimed streptavidin can be incorporated into a functioning product with no effect on stability or performance.

### Example 5

### Particle Size as an Indicator of Aggregation During Manufacture

As an initial quality control, the finished beads were sized to check for aggregation during manufacture. 5 uL of beads were mixed with 1 mL of diH2O in a disposable cuvette and read by the Anton Paar Litesizer™ 100 particle size analyzer after brief vortexing (5-10 seconds), mixing (10 minutes on mixer), and 30-60 seconds of sonication (if used). None of the three lots show aggregation from production pre- or post-sonication (Tables 3 and 4). On average, aggregate polydispersity is larger than monomeric polydispersity.

TABLE 3
Particle Sizing - No sonication in preparation

| | hydrodynamic | PEAK | polydispersity |
|---|---|---|---|
| Lot | dia | 1 | index |
| MSAv | 2355 | 2314 | 23.10% |
| FSAv | 1584.6 | 1025.8 | 142.80% |
| RSAv | 1449 | 1106.9 | 18.80% |

TABLE 4
Particle Sizing - With sonication in preparation

| | hydrodynamic | PEAK | polydispersity |
|---|---|---|---|
| Lot | dia | 1 | index |
| MSAv | 732 | 674.2 | 11.60% |
| FSAv | 703 | 631.5 | 4.30% |
| RSAv | 713 | 683.4 | 25.70% |

### Example 6

### Test for Biotin Leaching

To test for potentially problematic levels of biotin leaching, the biotin saturated and conjugated streptavidin coated beads (Biotinylated 100BS streptavidin-beads) were suspended in serum with a biotin concentration of less than 100 μg/ml. 400 μL of the serum was treated with 0.5 mg of beads (200 μL-2.5 mg/mL) for 10 minutes on a mixer at RT, magnetically separated according to the following protocol:
1. Remove the Biotinylated 100BS streptavidin-beads lot MSAv, FSAv, or RSAv reagent vial from storage and vortex for a minimum of 10 seconds at medium speed to mix well and resuspend the reagent.
2. Insert the reagent vial in the foam vial holder.
3. Insert an empty Micro tube 2 ml (SARSTEDT Order Number 72.694) into the VeraMag magnet until the collar of the tube contacts the magnet frame.
4. Dispense 200 μL of the well-mixed reagent (beads) into the empty tube to separate the reagent on the magnet for >30 seconds to form a reagent pellet.
5. Carefully aspirate and discard all of the storage buffer supernatant (˜200 μL) without disturbing the reagent pellet.
6. Dispense 400 μL of well-mixed serum or plasma sample into the tube containing the reagent pellet.
7. Tighten the screw cap on the tube, remove the tube from the magnet, and vortex for a minimum of 10 seconds at medium speed to mix well and resuspend the reagent in the sample.
8. Place the tube onto a laboratory mixer at medium speed and incubate at room temperature for 10 minutes.
9. Loosen and remove the screw cap and insert the tube into the magnet until the collar of the tube contacts the magnet frame.
10. Magnetically separate the reagent for >4 minutes to form a reagent pellet.
11. Carefully aspirate the sample supernatant without disturbing the reagent pellet and dispense the sample into a transfer tube for testing. Note: All of the sample supernatant (˜400 μL) can be aspirated if this step is performed carefully. If any of the reagent is accidentally aspirated then simply return the sample/reagent mixture to the tube and return to step 10.
12. The sample is now ready for testing.

The treated serum was tested on the IDK BIOTIN ELISA assay (Immundiagnostik AG). The IDK BIOTIN ELISA is a competitive immunoassay; biotin in the sample will reduce the signal generated. Concentration of biotin is determined by comparison to a calibration curve. A calibration curve was run with each of the three tested lots. In all cases biotin was detected at a level of less than 1200 μg/ml indicating that any leaching of biotin from the beads was at a level that would not cause heterophilic interference in standard immunoassays (Tables 5).

TABLE 5
Quantitation of Biotin in treated serum

| Sample | Abs 450 | Assigned pg/mL |
|---|---|---|
| Cal 1 | 2.5145 | 0 |
| Cal 2 | 2.198 | 75 |
| Cal 3 | 1.91 | 150 |
| Cal 4 | 1.3 | 300 |
| Cal 5 | 0.812 | 600 |
| Cal 6 | 0.533 | 1200 |
| RSAv QC1 | 0.597 | 1031 (<1200) |
| Cal 1 | 2.084 | 0 |
| Cal 2 | 1.789 | 75 |
| Cal 3 | 1.622 | 150 |
| Cal 4 | 0.981 | 300 |
| Cal 5 | 0.597 | 600 |
| Cal 6 | 0.423 | 1200 |
| FSAv QC1 | 1.298 | 220 (<1200) |
| Cal 1 | 2.899 | 0 |
| Cal 2 | 2.546 | 75 |
| Cal 3 | 2.302 | 150 |
| Cal 4 | 1.613 | 300 |
| Cal 5 | 0.941 | 600 |
| Cal 6 | 0.623 | 1200 |
| MSAv QC1 | 2.117 | 193 (<1200) |

### Example 7

### HPLC Depletion Assays

The three lots of biotin-saturated and -conjugated streptavidin-coated beads were used in depletion assays to access their ability to specifically remove anti-streptavidin and anti-biotin interferences, but not other interferences. To this end a series of 4 HPLC depletion studies were run:
1) An HPLC depletion assay using affinity-purified goat IgG and affinity-purified goat IgG conjugated to biotin in order to gauge specificity of the product for only anti-SAv and anti-Bt antibodies.
2) An HPLC depletion assay using anti-streptavidin antibody to quantitate binding capacity of the reagent.
3) An HPLC depletion assay using anti-biotin antibody will be performed to quantitate binding capacity of the reagent.
4) An HPLC depletion assay using a mix of A) anti-biotin antibody and anti-streptavidin antibody, and B) anti-streptavidin antibody and affinity-purified goat IgG, to demonstrate the multiplexing capability and specificity of the reagent.

Concentrations of the various antibodies (Ab) and antibody-biotin (Ab-Bt) conjugates, in phosphate-buffered saline (PBS), pH 7.4, were determined. For each sample 200 μl of the biotin saturated and conjugated streptavidin coated bead suspension (2.5 mg/ml) was aliquoted into a tube, magnetically separated, and the storage buffer removed. 400 μl of each Ab or Ab-Bt conjugate was added to a bead-containing tube, vortexed and incubated on a mixer for 10 minutes. The beads were again magnetically separated and the supernatant of treated sample was drawn off and loaded into a micro tube insert for HPLC tubes for size exclusion chromatography (SEC) analysis. The detailed protocol for the pre-treatment was as follows:
1. Remove the Biotinylated 100BS streptavidin-beads lot MSAv, FSAv, or RSAv reagent vial from storage and vortex for a minimum of 10 seconds at medium speed to mix well and resuspend the reagent.
2. Insert the reagent vial in the foam vial holder.
3. Insert an empty Micro tube 2 ml (SARSTEDT Order Number 72.694) into the VeraMag magnet until the collar of the tube contacts the magnet frame.
4. Dispense 200 μL of the well-mixed reagent (beads) into the empty tube to separate the reagent on the magnet for >30 seconds to form a reagent pellet.
5. Carefully aspirate and discard all of the storage buffer supernatant (˜200 μL) without disturbing the reagent pellet.
6. Dispense 400 μL of well-mixed serum or plasma sample into the tube containing the reagent pellet.
7. Tighten the screw cap on the tube, remove the tube from the magnet, and vortex for a minimum of 10 seconds at medium speed to mix well and resuspend the reagent in the sample.
8. Place the tube onto a laboratory mixer at medium speed and incubate at room temperature for 10 minutes.
9. Loosen and remove the screw cap and insert the tube into the magnet until the collar of the tube contacts the magnet frame.
10. Magnetically separate the reagent for >4 minutes to form a reagent pellet.
11. Carefully aspirate the sample supernatant without disturbing the reagent pellet and dispense the sample into a transfer tube for testing. Note: All of the sample supernatant (˜400 μL) can be aspirated if this step is performed carefully. If any of the reagent is accidentally aspirated then simply return the sample/reagent mixture to the tube and return to step 10.
12. The sample is now ready for testing.

The untreated antibodies were also run on SEC as controls. SEC buffer was 50 mM Potassium Phosphate, 250 mM Potassium Chloride, pH 6.8, and was pumped at 1 ml/min for 20 minutes on a G4000 column 7.8 mm×30 cm, using an injection of 5 μg/100 μl. Absorbance at 220 nm and 280 nm was monitored and the A280 used for peak analysis. Results are shown in Table 6.

TABLE 6
Quantitation of depletion and bead capacity

| | | | | | | | | μg Ab |
|---|---|---|---|---|---|---|---|---|
| | | TREATED | UNTREATED | | | μg Ab | mg of | depleted |
| BEAD | | PEAK | PEAK | % | % | in | beads | per mg |
| LOT | SAMPLE | AREA | AREA | remaining | depleted | rxn | in rxn | beads |
| MSAv | α-SAv Ab¹ | 427.65 | 1267.02 | 33.75% | 66.25% | 46.8 | 1 | 31 |
| | α-Bt Ab² | 1055.61 | 1419.01 | 74.39% | 25.61% | 46.8 | 1 | 12 |
| | AP Goat Ab³ | 1331.92 | 1376.82 | 96.74% | 3.26% | 48.4 | 1 | 1.6 |
| | AP goat IgG-Biotin⁴ | 1005 | 1006 | 99.90% | 0.10% | 40 | 1 | 0 |
| | α-SAv/α-Bt mix⁵* | 81 | 436 | 18.58% | 81.42% | 23.4 | 1 | 19.1 |
| | α-SAv/AP goat IgG | 319 | 590 | 54.07% | 45.93% | 23.8 | 1 | 10.9 |
| | mix⁶* | | | | | | | |
| FSAv | α-SAv Ab | 395 | 1392 | 28.38% | 71.62% | 46.8 | 1 | 33.5 |
| | α-Bt Ab | 378 | 1424 | 26.54% | 73.46% | 46.8 | 1 | 34.4 |
| | AP Goat Ab | 1294 | 1398 | 92.56% | 7.44% | 48.4 | 1 | 3.6 |
| | AP goat IgG-Biotin | 995 | 1006 | 98.91% | 1.09% | 40 | 1 | 0.4 |
| RSAv | α-SAv Ab | 521 | 1267 | 41.12% | 58.88% | 46.8 | 1 | 27.6 |
| | α-Bt Ab | 786 | 1424 | 55.20% | 44.80% | 46.8 | 1 | 21 |
| | AP Goat Ab | 1353 | 1398 | 96.78% | 3.22% | 48.4 | 1 | 1.6 |
| | AP goat IgG-Biotin | 1006 | 1006 | 100.00% | 0.00% | 40 | 1 | 0 |

(“α-” symbolizes “anti”, “AP” symbolizes “affinity purifies antibody”; “Bt” symbolized “biotinylated antibody”)
¹anti-streptavidin antibody
²anti-biotin antibody
³affinity purified goat IgG
⁴affinity purified goat IgG-biotin conjugate
⁵mixture of anti-streptavidin antibody and anti-biotin antibody
⁶mixture of anti-streptavidin antibody and affinity purified goat IgG
*Peak areas of individual fractions for multiplex samples: 5.85 μg of anti-biotin antibody - area 134; 6.05 μg affinity purified goat IgG - area 289; 5.85 μg anti-streptavidin antibody - area 243.

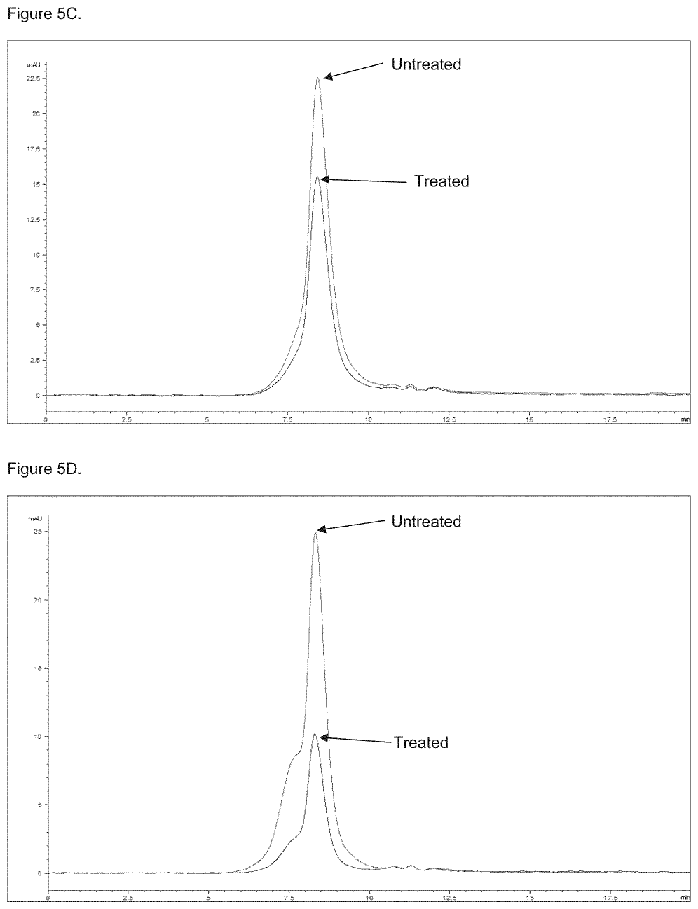

Affinity-purified Goat IgG was run as a control to establish background depletion as the beads are intended to be neutral to antibodies not specifically anti-biotin or anti-streptavidin. All three lots show minimal to no binding of this control (92.56%-96.78% of antibody still present after treatment; FIG. 5A depicts the results with the MSAv lot).

Affinity-purified Goat IgG conjugated to Biotin, was run as a further control to establish background depletion as, again, the beads are intended to be neutral to antibodies not specifically anti-biotin or anti-streptavidin, even if biotinylated. All three lots show minimal to no binding of this control (98.91%-100% of antibody present after treatment; FIG. 5B depicts the results with the MSAv lot).

All three lots show greater than 10 μg depletion per mg of beads of the anti-biotin antibody (12, 34.4, and 21 μg/mg depletion; see Table 6; FIG. 5C depicts the results with the MSAv lot). All three lots show greater than 20 μg depletion per mg of beads of the anti-SAv antibody (31, 33.5, and 27.6 μg/mg depletion; see Table 6; an exemplary profile is shown in FIG. 5D, which depicts the results with the MSAv lot).

A mix of the anti-Streptavidin and anti-Biotin antibodies was tested using the MSAv lot of beads to demonstrate the multiplexing capability of the biotin saturated and conjugated streptavidin-coated beads. Also, a mix of the anti-Streptavidin and AP goat IgG antibodies was tested to demonstrate specificity of binding by the reagent. The data illustrates that the biotin saturated and conjugated streptavidin-coated beads depletes both of the anti-Streptavidin and anti-Biotin antibodies when present in tandem (19.1 μg depleted out of 23.4 μg present) and that the product specifically removes only the anti-streptavidin antibody when mixed with AP goat IgG (10.9 μg depleted out of 23.8 μg present). As previous data showed no binding of AP goat IgG individually (97% of Ab still present after treatment), it can be safely inferred that the depletion of roughly half of the mix (46%) illustrates only the anti-streptavidin Ab was depleted.

### Example 8

### Impact of Treatment on Analyte Detection

The neutrality of the biotin-saturated and conjugated streptavidin-coated beads (Biotinylated 100BS streptavidin-beads) was tested in a commercial assay for serum parathyroid hormone. The DRG PTH Intact ELISA (DRG International, Inc., Part Number EIA3645) is a sandwich ELISA assay using two different goat anti-PTH polyclonal antibodies recognizing distinct portions of the hormone. One of the antibodies is biotinylated and serves as the capture reagent and the other is conjugated to horseradish peroxidase and serves as the detection reagent.

400 μl each of two serum samples (QC1 and QC3) were treated with 0.5 mg of beads (200 ul at 2.5 mg/mL) for 10 minutes on a mixer at RT, magnetically separated, according to the following protocol:
1. Remove the Biotinylated 100BS streptavidin-beads lot SAv, FSAv, or RSAv reagent vial from storage and vortex for a minimum of 10 seconds at medium speed to mix well and resuspend the reagent.
2. Insert the reagent vial in the foam vial holder.
3. Insert an empty Micro tube 2 ml (SARSTEDT Order Number 72.694) into the VeraMag magnet until the collar of the tube contacts the magnet frame.
4. Dispense 200 μL of the well-mixed reagent (beads) into the empty tube to separate the reagent on the magnet for >30 seconds to form a reagent pellet.
5. Carefully aspirate and discard all of the storage buffer supernatant (˜200 μL) without disturbing the reagent pellet.
6. Dispense 400 μL of well-mixed serum or plasma sample into the tube containing the reagent pellet.
7. Tighten the screw cap on the tube, remove the tube from the magnet, and vortex for a minimum of 10 seconds at medium speed to mix well and resuspend the reagent in the sample.
8. Place the tube onto a laboratory mixer at medium speed and incubate at room temperature for 10 minutes.
9. Loosen and remove the screw cap and insert the tube into the magnet until the collar of the tube contacts the magnet frame.
10. Magnetically separate the reagent for >4 minutes to form a reagent pellet.
11. Carefully aspirate the sample supernatant without disturbing the reagent pellet and dispense the sample into a transfer tube for testing. Note: All of the sample supernatant (˜400 μL) can be aspirated if this step is performed carefully. If any of the reagent is accidentally aspirated then simply return the sample/reagent mixture to the tube and return to step 10.
12. The sample is now ready for testing.

The treated serum was then tested on the DRG PTH ELISA assay. QC1 is an in-house QC sample with less than 100 μg/mL Biotin (which will not affect the PTH assay mechanics) and roughly 190 μg/mL PTH. QC3 is an in-house QC sample with about 250,000 μg biotin/mL (which will affect the PTH assay mechanics—assay yields severely depressed results) and roughly 190 μg/mL PTH. QC1 serum treated with each of the different lots show no significant deviation in PTH results (100.6, 101.2, and 106.1% detection) and QC3 samples all yield severely depressed results as expected since 100BS streptavidin-beads will not bind free biotin (Table 7). These data demonstrate the neutrality of the 100BS streptavidin-beads beads.

TABLE 7
PTH ELISA data for neutrality

| | Average | Dose | Sample/Neat |
|---|---|---|---|
| Sample | A450 | pg/mL | untreated |
| QC1 untreated | 0.9395 | 173.2 | Baseline |
| QC1 v MSAv | 0.9445 | 174.3 | 100.6% |
| QC1 v FSAv | 0.9495 | 175.4 | 101.2% |
| QC1 v RSAv | 0.989 | 183.8 | 106.1% |
| QC3 untreated | 0.299 | 21.7 | 12.5% |
| QC3 v MSAv | 0.2895 | 19.4 | 11.2% |
| QC3 v FSAv | 0.297 | 21.2 | 12.2% |
| QC3 v RSAv | 0.3095 | 24.1 | 13.9% |

Analyte detection after treatment of samples containing anti-streptavidin and anti-biotin antibody interferents was determined. This was accomplished by spiking affinity purified anti-streptavidin and anti-biotin goat antibodies into serum QC1 and comparing analyte detection results from treated and untreated samples.

A 400 μl aliquot of each serum sample (QC1, and QC1 spiked with anti-biotin antibody to 16.5 μg/mL) was treated with differing amounts of the beads (100 to 400 μl at 2.5 mg/mL) from all three lots for 10 minutes on a mixer at RT, magnetically separated, and the treated serum was tested on the DRG PTH ELISA assay. QC1 is an in-house QC sample with less than 100 μg/mL Biotin (which will not affect the PTH assay mechanics) and roughly 190 μg/mL PTH. The concentration of anti-biotin antibody spiked into QC1 interferes with the PTH assay mechanics, causing severely depressed results.

MSAv beads were able to successfully deplete all anti-Bt Antibody (anti-Bt Aby) and restore correct PTH result with 1 mg of beads per 200 μl of sample (with 16.5 μg/mL anti-Bt Aby conc.). FSAv and RSAv were able to achieve the same result with far less: 0.25 mg for FSAv and 0.375 mg for RSAv. The QC1 sample spiked with anti-Bt Aby and NOT treated with Biotinylated 100BS streptavidin-beads yielded severely depressed results, only 2.7% of control, in line with expectation (Tables 8 and 9). The lower capacity of the MSAv lot is consistent with the results observed in Example 7 (above).

TABLE 8
Analyte detection in serum spiked with anti-biotin
Ab and treated with beads from lot MSAv

| | Average | Dose result | |
|---|---|---|---|
| Sample | A450 | pg/mL | |
| | | | Sample/Neat untreated |
| QC1 Neat (control) | 0.926 | 179.8 | 100.00% |
| QC1 anti-Bt Aby | 0.304 | 8.4 | 4.70% |
| spike (neg control) | | | |
| QC1 PBS spike | 0.853 | 158.4 | 88.10% |
| (baseline control) | | | |
| | | | Treated/QC1 PBS spike |
| QC1 spike 0.25 mg, | 0.456 | 69.4 | 43.81% |
| 10 min treated | | | |
| QC1 spike 0.25 mg, | 0.467 | 72 | 45.45% |
| 30 min Treated | | | |
| QC1 spike 0.375 mg | 0.571 | 91.2 | 57.58% |
| Treated | | | |
| QC1 spike 0.5 mg | 0.721 | 121.7 | 76.83% |
| Treated | | | |
| QC1 spike 1.0 mg | 0.855 | 158.9 | 100.32% |
| Treated | | | |

TABLE 9
Analyte detection in serum spiked with anti-Bt Abv
and treated with beads from lots FSAv and RSAv

| | Average | Dose result | Treated/PBS |
|---|---|---|---|
| Treated Samples | A450 | pg/mL | spike untreated |
| FSAv 0.25 mg treatment | 0.779 | 200.2 | 108.3% |
| FSAv 0.375 mg treatment | 0.779 | 200.1 | 108.2% |
| FSAv 0.5 mg treatment | 0.714 | 182.3 | 98.6% |
| FSAv 1 mg treatment | 0.743 | 190.3 | 102.9% |
| RSAv 0.25 mg treatment | 0.649 | 163.3 | 88.3% |
| RSAv 0.375 mg treatment | 0.742 | 190.2 | 102.8% |
| RSAv 0.5 mg treatment | 0.756 | 193.8 | 104.8% |
| RSAv 1 mg treatment | 0.725 | 185.5 | 100.3% |
| Control | Average | Dose result | Treated/QC1 |
| Samples untreated | A450 | pg/mL | PBS spike |
| QC1 NEAT | 0.720 | 184.1 | 99.6% |
| QC1 w/anti-biotin Aby | 0.272 | 5.0 | 2.7% |
| QC1 w/PBS pH 7.5 (control) | 0.723 | 185.0 | 100.0% |

Similarly, a 200 μl aliquot of each serum sample (QC1, and QC1 spiked with anti-streptavidin Aby (anti-SAv Aby) to 16.5 μg/mL, or anti-SAv Aby/anti-Bt Aby multiplex mixture to 16.5 μg/mL, 8.25 μg/mL each), was treated with 100 μl of beads at 2.5 mg/mL for 10 minutes on a mixer at RT, magnetically separated, and the treated serum was tested on the DRG PTH ELISA assay. As noted previously, QC1 is an in-house QC sample with less than 100 μg/mL Biotin (which will not affect the PTH assay mechanics) and roughly 190 μg/mL PTH. The anti-SAv Aby and anti-Bt Aby interfere with the PTH assay mechanics, causing severely depressed results.

At the concentration used (16.5 μg/mL) anti-SAv Aby caused severely depressed results, as expected (29% of baseline). The multiplex mixture did as well (21% of baseline). Beads from lot RSAv were able to successfully deplete anti-SAv Aby and begin to restore PTH detection with 0.25 mg of beads per 200 μl of sample resulting in readings of up to 82% of the baseline value. Increasing the amount of bead used in the treatment to 0.5 mg per 200 μl of this sample restored the value to 104% of baseline. Beads from lot FSAv at 0.25 mg of beads per 200 μl of sample restored the PTH level to 91% of baseline. Beads from lot MSAv at 0.375 mg per 200 μl of this sample restored the PTH value to 92% of baseline. All lots were able to successfully restore correct PTH result with 0.5 mg of beads (RSAv—104%; FSAv—101%; MSAv 100%) (Table 10).

TABLE 10
Analyte detection in serum spiked with anti-SAv Abv, or anti-SAv
Abv and anti-Bt Abv, and treated with beads from all three lots

| | avg | | % of |
|---|---|---|---|
| Sample (200 uL volume) | A450 | spline | baseline |
| QC1 + 100 uL RSAv (0.25 mg) | 0.6435 | 114.35 | 82% |
| QC1 + 150 uL RSAv (0.375 mg) | 0.6835 | 124.32 | 89% |
| QC1 + 200 uL RSAv (0.5 mg) | 0.7765 | 145.69 | 104% |
| QC1 + 400 uL RSAv (1.0 mg) | 0.872 | 165.46 | 119% |
| QC1 + 100 uL FSAv (0.25 mg) | 0.695 | 127.09 | 91% |
| QC1 + 150 uL FSAv (0.375 mg) | 0.809 | 152.63 | 109% |
| QC1 + 200 uL FSAv (0.5 mg) | 0.753 | 140.51 | 101% |
| QC1 + 400 uL FSAv (1.0 mg) | 0.715 | 131.82 | 94% |
| QC1 + 100 uL MSAv (0.25 mg) | 0.6735 | 121.87 | 87% |
| QC1 + 150 uL MSAv (0.375 mg) | 0.6975 | 127.69 | 92% |
| QC1 + 200 uL MSAv (0.5 mg) | 0.746 | 138.94 | 100% |
| QC1 + 400 uL MSAv (1.0 mg) | 0.628 | 110.41 | 79% |
| Multi + 100 uL RSAv (0.25 mg) | 0.7265 | 134.49 | 96% |
| multi + 100 uL FSAv (0.25 mg) | 0.6845 | 124.56 | 89% |
| multi + 100 uL MSAv (0.25 mg) | 0.6925 | 126.49 | 91% |
| QC1 + 300 uL MSAv (0.75 mg) | 0.7455 | 138.82 | 100% |
| QC1 + 300 uL RSAv (0.75 mg) | 0.737 | 136.90 | 98% |
| QC1 + 300 uL FSAv (0.75 mg) | 0.692 | 126.37 | 91% |
| | avg | | % of |
| Control samples (200 uL) | A450 | spline | baseline |
| QC1 Multiplex Spike | 0.328 | 29.48 | 21% |
| QC1 PBS | 0.7485 | 139.50 | 100% |
| QC1 neat | 0.8315 | 157.30 | 113% |
| QC1 anti-SAv Aby spike | 0.3655 | 40.17 | 29% |

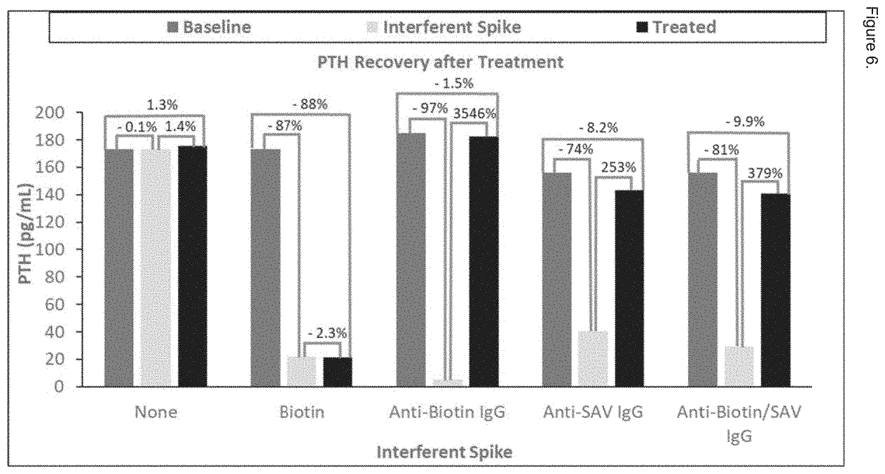

A summary of the results of the above PTH detection experiments using lot MSAv is presented in FIG. 6. The Biotinylated 100BS streptavidin-beads had no effect when no interferent was used or when biotin was the interferent, but was effective in removing anti-biotin and anti-streptavidin interferents, individually or mixed together.

Specifically, in the left-most group of bars in FIG. 6, labeled “None”, there was no interferent added to the interferent spike sample, that is, it was a re-run of the Baseline sample, and the concentration of PTH detected differed by only −0.1%. Treatment of the Baseline sample with biotin-saturated and -conjugated streptavidin-coated beads, without any interference (None), was only +1.3% different than the Baseline sample and only +1.4% different than the Baseline sample re-run (no interference spike). These results are well with-in the precision profile of this PTH ELISA and demonstrate reagent neutrality and that treatment of the sample did not introduce any dilution or matrix effect. The Biotin spike caused significant interference in this PTH ELISA assay and resulted in a 87% decrease in detection. When the Biotin spiked sample was treated with the beads, the result did not significantly change and was only −2.3% different, was also 88% lower than the Baseline result. This is expected as Biotinylated 100BS streptavidin-beads do not bind free biotin and will not mitigate this interference mechanism. The Anti-Bt Aby spike caused significant interference in this PTH ELISA assay and resulted in a 97% decrease in detection. When the Anti-Bt Aby spike was treated the result significantly changed and was +3,546% different, but was only −1.5% different than the Baseline result. This is expected as the Biotinylated 100BS streptavidin-beads were designed to bind and deplete anti-biotin interference and report an accurate result similar to the Baseline result without Anti-biotin interference. The Anti-SAv Aby spike also caused significant interference in this PTH ELISA assay and resulted in a 74% decrease in detection. When the Anti-SAv Aby spike was treated the result significantly changed and was +253% different, but was only −8.2% different than the Baseline result. This is expected as the Biotinylated 100BS streptavidin-beads were designed to bind and deplete anti-streptavidin interference and report an accurate result similar to the Baseline result without Anti-SAv Aby interference. Finally, a 1:1 mixture Anti-SAv Aby and Anti-Bt Aby spike caused significant interference in this PTH ELISA assay and resulted in an 81% decrease in detection. When the Anti-SAv Aby and Anti-Bt Aby spike was treated, the result significantly changed and was +379% different, but was only −9.9% different than the Baseline result. These results are still with-in the precision profile of this PTH ELISA. This is expected as the Biotinylated 100BS streptavidin-beads were designed to bind and deplete both anti-Biotin and anti-streptavidin interferences and report an accurate result similar to the Baseline result without Anti-biotin and Anti-streptavidin interferences. These data also demonstrate the ability of the Biotinylated 100BS streptavidin-beads to simultaneously bind and deplete both interference mechanisms from the same sample.

### Example 9

### Biotin Saturation of Soluble Streptavidin

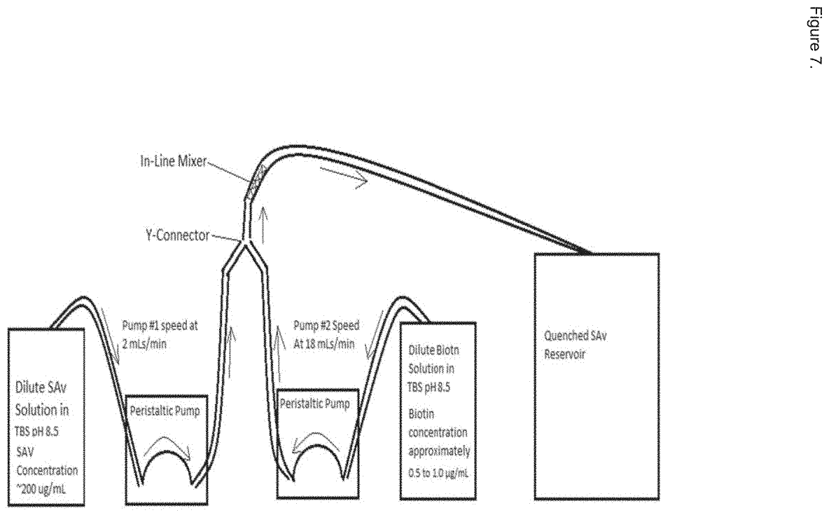

A dilute streptavidin (SAv) (or modified SAv or blocked SAv) solution in tris buffered saline, pH 8.5 (TBS) is prepared at approximately 200 μg SAv/mL. A dilute solution of biotin in TBS is also prepared at approximately 0.50-1.00 μg biotin/mL. The two solutions are metered together and mixed in line at a ratio of 1 Volume SAv/TBS to 9 Volumes Biotin/TBS by, for example, pumping through silicon tubing (such as PN 96440-13 (Cole Parmer)) meeting at a Y-connector (such as, Masterflex PN 30614-08 (Cole Parmer)) and immediately mixing with an in-line mixer (such as, In-line mixer PN HT-40-3.18-12-PP (StaMixCo)) in the outlet tubing (FIG. 7). The SAv solution can be pumped at 2 mL/min and the biotin solution can be pumped at 18 mL/min using, for example, peristaltic pumps (such as, Masterflex EZIoad2 model 07522-20 (Cole Parmer)). The solution containing the biotin and streptavidin is mixed for 30 to 120 minutes.

Other concentrations of biotin and SAv, other buffer systems, and other pump speeds may be used, but the ratio of biotin concentration to SAv concentration and metered addition ratio should be maintained. The nine volumes of biotin solution should contain 7.4 moles of biotin for every mole of streptavidin in the SAv solution. Note that this is a somewhat higher biotin to streptavidin ratio than used in minimal saturation procedure for the bead-conjugated SAv. Taking the molecular weight of streptavidin as 52000, 300 mL of 200 μg/ml SAv solution contains 1.1538 μmoles of streptavidin. Taking the molecular weight of biotin as 244.31, 8.53072 μmoles of biotin (for the 7.4:1 molar ratio) is 2084 μg of biotin, so that in nine volumes the biotin concentration is 772 ng/mL.

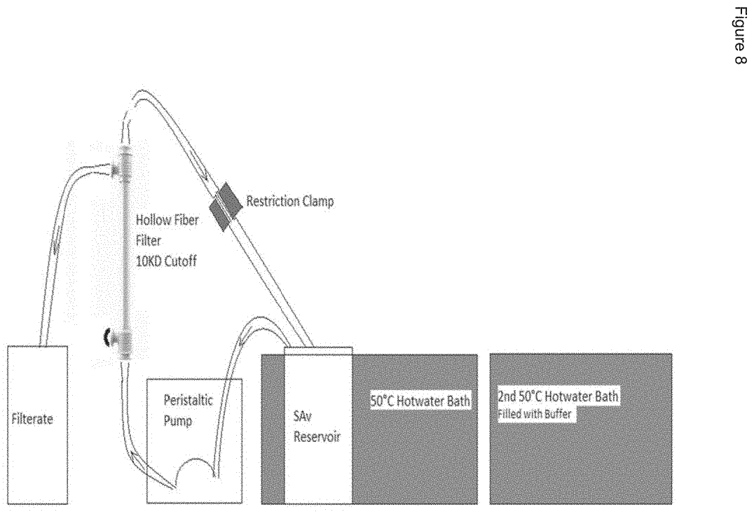

To remove unbound and non-specifically bound biotin, the biotin-saturated streptavidin was subjected to diafiltration and washing. A hot water bath was are filled with purified water and heated to 50° C. A second hot water bath was filled with a buffer of 10 mM tris, 50-150 mM NaCl, and heated to 50° C. (Alternatively, this buffer could also contain 0.01-1% w/v TWEEN 20 or other surfactant). The reservoir containing the biotin-saturated streptavidin was placed into the first hot water bath. Using a hollow fiber filter such as a MiniKros Sampler Hollow Fiber Filter with 10 kD molecular weight cutoff (Repligen; PN 504-E010-05-N; mPes; 0.5 mm), tubing was attached to in-line flow ports (top and bottom) and one side port. The second side port was capped. The side port tubing led filtrate to a waste container. Tubing leading from the reservoir proceeded through a peristaltic pump to the hollow fiber filter. Tubing leading from the in-line flow outlet port conveyed retentate back to the reservoir (FIG. 8). When the water baths and reservoir had reached 50° C. the peristaltic pump was turned on and a clamp applied to the retentate tubing creating a back pressure so that filtration occurred, reducing the volume of the biotin-saturated streptavidin solution and concentrating the protein. Retentate flow was about 360 ml/min and filtrate flow was about 95 ml/min. When the reservoir reached about 15% (or less) of its original volume, buffer from the 2nd water bath was added to the reservoir to restore the original volume. (A sample for quality control was taken just before volume restoration). Filtration and restoration of volume were repeated for a total of at least 5 times; additional wash cycles may be added if desired. Following the final restoration of volume the retentate was concentrated to about 10% of the original volume (other volumes could be used, as desired). Free biotin in the final retentate should be less than 1200 μg/ml. The concentrated biotin-saturated streptavidin was filtered through a 0.2 μm filter.

Free biotin in the presence of solubilized streptavidin is considered evidence for a completed biotin quench process. However, free biotin can itself cause an interference and is therefore undesirable in a blocking reagent to block streptavidin and other interferences, and therefore needs to be removed. The retentate after each wash and the final retentate (in duplicate) were assayed for free biotin content using an ELISA Biotin Assay (Immundiagnostik, PN KR8141) to obtain the results shown in Table 11.

TABLE 11
Quantitation of free biotin in QSAv preparation
quality control samples (and calibration)

| | | Cals | Results |
|---|---|---|---|
| ID | OD450 | pg/mL | pg/mL |
| Cal 1 | 2.287 | 0 | 0 |
| Cal 2 | 2.388 | 75 | 75 |
| Cal 3 | 2.095 | 150 | 150 |
| Cal 4 | 1.419 | 300 | 300 |
| Cal 5 | 0.918 | 600 | 600 |
| Cal 6 | 0.354 | 1200 | 1200 |
| Ctl A | 1.652 | | 238 |
| Ctl B | 1.031 | | 514 |
| Quench 19 ug/mL | 0.302 | | >1200 |
| 1st retentate | 0.307 | | >1200 |
| 2nd retentate | 0.338 | | >1200 |
| 3rd retentate | 0.377 | | 1170 |
| 4th retentate | 0.347 | | >1200 |
| 5th retentate | 0.428 | | 1105 |
| Final | 0.836 | | 670 |
| Final | 0.876 | | 635 |

The final retentate contained less than 700 μg/mL of free biotin, substantially below the less than 1200 μg/mL requirement. The final retentate had a concentration of 201 μg streptavidin/mL so that it contained ˜3.16-3.33 pg free biotin/μg streptavidin.

A further step to remove any incompletely quenched streptavidin may be added. 2-iminobiotin conjugated to agarose (Sigma Aldrich PN I4507-5ML) can be used for this purpose. 2-iminobiotin reversibly binds to SAv under alkaline conditions. Binding is strongest at pH 10 to 11. SAv will be released under acidic conditions such as at pH 4.0. SAv already quenched with biotin should not bind. Therefore, the flow through of quenched SAv from a column of alkaline 2-iminobiotin-agarose should be only biotin quenched SAv. SAv not biotin quenched should adhere to the column. The column may be regenerated for subsequent reuse with a pH 4 buffer.

### Example 10

### Removal of Goat Anti-Mouse Antibodies with Streptavidin Beads Conjugated with Mouse IgG

Magnetic nanoparticles (beads) of 500 to 600 nm diameter were coated with streptavidin. Two affinity purified mouse IgG preparations were biotinylated covalently with NHSester-Peg(4)-Biotin. Mouse IgG #1 was a polyclonal non-specific mouse IgG. Mouse IgG #2 was a monoclonal mouse IgG. When the beads were exposed to the biotinylated mouse IgGs, approximately 30 μg of IgG adhered to each mg of beads. Beads were made with just the polyclonal mouse IgG, with just the monoclonal mouse IgG, and with a mixture of the two mouse IgG preparations. Those mouse antibodies serve as a capture moiety for any anti-mouse IgG antibodies, either specific affinity purified or heterophilic, to which they will be exposed. These mouse IgG-conjugated streptavidin bead were then used to clean a sample (in this case buffer) which had been spiked with affinity purified goat anti-mouse antibody (Lampire Biological Laboratories). An aliquot of the sample was then analyzed by HPLC size exclusion chromatography for IgG content.

A possible concern with using a biotinylation to anchor a capture reagent on the streptavidin is that the affinity of the biotin can be less than that of free biotin to the extent that the capture reagent dissociates from the streptavidin in the course of the cleaning procedure. To check for this, the cleaning procedure was carried out normally and in the presence of 20 μg/mL free biotin (a >200-fold molar excess over the streptavidin). If the biotinylated IgG dissociates from the streptavidin-coated bead it will not be able to rebind in the presence of the excess free biotin, and as a consequence the amount of goat anti-mouse antibody removed with the bead will be decreased. Alternatively, if dissociation of the biotinylated IgG is not occurring to a significant degree, then the amount of goat anti-mouse IgG will not vary significantly between the samples with and without free biotin.

Specifically, one aliquot of each of the three mouse IgG conjugated streptavidin beads (with IgG #1, IgG #2, and a mixture of the two) was magnetically separated from storage buffer (TBS) and mixed with TBS containing 20 μg/ml biotin. Goat anti-mouse IgG was diluted to 200 μg/mL in a) TBS and b) TBS containing 20 μg/ml biotin. Two aliquots of each of the three mouse IgG conjugated streptavidin beads—one biotin exposed, the other not—were magnetically separated from their buffer. Using 2.5 mg of beads per mL of goat anti-mouse IgG, the goat anti-mouse IgG in TBS was added to the beads that had not been exposed to biotin, and the goat anti-mouse IgG in TBS containing 20 μg/ml biotin was added to the beads that had been exposed to biotin. These mixtures were vortexed for 10 seconds to suspend the beads and mixed overnight. The resulting mixtures had the beads magnetically separated and the supernatant samples s were then analyzed by HPLC-SEC using the area under the curve for absorbance at 280 nm for the peak corresponding to goat IgG. Results are shown in Table 12.

TABLE 12
Removal of goat anti-mouse IgG

| | | μg Goat anti-Mouse |
|---|---|---|
| | Area under IgG | Depleted per mg of |
| Sample (without biotin) | Peak 280 nm | beads |
| 200 μg/mL Goat Anti-Mouse | 1020 | |
| (control) | | |
| Polyclonal mouse IgG beads | 299 | 56.5 |
| Monoclonal mouse IgG beads | 688 | 26.0 |
| Mixed polyclonal and | 387 | 49.6 |
| monoclonal mouse IgG beads | | |
| | | μg Goat anti-Mouse |
| | Area under IgG | Depleted per mg of |
| Sample (with biotin) | Peak 280 nm | beads |
| 200 μg/mL Goat Anti-Mouse | 1002 | |
| (control) | | |
| Polyclonal mouse IgG beads | 254 | 60.1 |
| Monoclonal mouse IgG beads | 624 | 31.1 |
| Mixed polyclonal and | 304 | 56.2 |
| monoclonal mouse IgG beads | | |

These data demonstrated that the mouse IgG-conjugated streptavidin beads were able to remove 26.0-60.1 μg of anti-mouse antibody per mg of bead. These data further demonstrated that the attachment of the biotinylated capture reagents (the mouse IgGs) was stable under the conditions of the cleaning procedure, even in the presence of a large excess of free biotin. Finally, these data demonstrated that these procedures are effective with capture moieties that are polyclonal and monoclonal antibodies, as well as a mixture of the two.

### Example 11. Removal of Biotin with Streptavidin Beads Conjugated with Mouse IgG

The three mouse IgG conjugated streptavidin bead preparations described above in Example 10 (polyclonal mouse IgG, monoclonal mouse IgG, and mix of polyclonal and monoclonal mouse IgG) were also tested for their ability to remove free biotin from a sample. Using essentially the same protocols described above, 0.75 mg of beads were combined with 200 μL of QC3, an in-house QC sample with about 250,000 μg biotin/mL, 50 ng biotin, incubated for 10 minutes, magnetically separated for 5 minutes, and the sample supernatant collected. The treated samples were tested on the IDK BIOTIN ELISA assay (Immundiagnostik AG; see Example 6). All three bead preparations were able to remove at least 49.7 ng biotin from 0.200 mL of QC3 at 250 ng biotin/mL (50 ng biotin total), or 66.3 ng of biotin/mg of bead, under these conditions and reduce the biotin concentration from 250,000 μg/mL to less than 300 μg/ml.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### REFERENCES

1. Society to Improve Diagnosis in Medicine (SIDM); https://betterdiagnosis.org/2.
2. Frequency that Laboratory Tests Influence Medical Decisions; http://jalm.aaccjnls.org/content/1/4/410
3. Frame, I. J., et al. (2019) Susceptibility of Cardiac Troponin Assays to Biotin Interference.

Am J Clin Pathol. 151: 486-493.
4. Wild, David. (2018) Biotin interference: answering questions, reducing the risk.

http://captodayonline.com/biotin-interference-answering-questions-reducing-the-risW
5. Katzman, B. M., et al. (2018) Prevalence of biotin supplement usage in outpatients and plasma biotin concentrations in patients presenting to the emergency department. Clinical Biochemistry. 60:11-16
6. Colon, P. J., Green, D. N. (2018) Biotin Interference in Clinical Immunoassays. JALM. 02(06): 941-951.
7. Kirkwood, Julie. (2018) Meeting the Biotin Challenge. Clinical Laboratory News. January, 2018.
8. Chun, Kelly Y. (2017) Biotin Interference in Diagnostic Tests. Clinical Chemistry. 63:619-620
9. Samarasinghe, S., et al. (2017). Biotin Interference with Routine Clinical Immunoassays: Understand the Causes and Mitigate the Risks. Endocr Pract. 23(8): 989-998.
10. Lam L., Kyle C. V. (2017). A simple method to detect biotin interference on immunoassays. Clin Chem Lab Med. 55(6): e104-e106.
11. Trambas C., et al. (2017). Depletion of biotin using streptavidin-coated microparticles: a validated solution to the problem of biotin interference in streptavidin-biotin immunoassays. Ann Clin Biochem. 55(2): 216-226.
12. Piketty, M. L., et al. (2017) High-dose biotin therapy leading to false biochemical endocrine profiles: validation of a simple method to overcome biotin interference. Clin. Chem. Lab Med. 55(6):817-825.
13. Barbesino, G. (2016) The Unintended Consequences of Biotin Supplementation: Spurious Immunoassay Results Lead to Misdiagnoses. Clinical Laboratory News, Bench Matters, December: 1-3.
14. The FDA Warns that Biotin May Interfere with Lab Tests: FDA Safety Communication; https://www.fda.gov/medical-devices/safety-communications/fda-warns-biotin-may-interfere-lab-tests-fda-safety-communication
15. Testing for Biotin Interference in In Vitro Diagnostic Devices;

https://www.fda.gov/regulatory-information/search-fda-guidance-documents/testing-biotin-interference-vitro-diagnostic-evices
16. Tytgat, H. L. P., et al. (2015). Endogenous biotin binding proteins: an overlooked factor causing false positives in streptavidin based protein detection. Microb Biotechnol. 8(1): 164-168.
17. Chen, T., et al. (2012). Biotin IgM Antibodies in Human Blood: A Previously Unknown Factor Eliciting False Results in Biotinylation-Based Immunoassays. PLoS One. 7(8): e42376.
18. Favresse, J., et al., (2018). Interferences with thyroid function immunoassays: clinical implications and detection algorithm. Endocrine Reviews. 39(5): 830-850.
19. Favresse J., et al. (2018). Antistreptavidin antibodies mimicking heterophilic antibodies in thyroid function tests. Clin Chem Lab Med. 56(7): e160-e163.
20. Lam, L. et al. (2018). Apparent Hyperthyroidism Caused by Biotin-Like Interference from IgM Anti-Streptavidin Antibodies. Thyroid. 28(8):1063-1067
21. Harsch I. A., et al. (2017). Implausible elevation of peripheral thyroid hormones during therapy with a protein supplement. Clin Chem Lab Med. 55(9): e197-e198.
22. Peltier L., et al. (2016). Anti-streptavidin interferences in Roche thyroid immunoassays: a case report. Clinical chemistry and laboratory medicine. 54(1): e11-14.
23. Rulander N.J., et al. (2013). Interference from antistreptavidin antibody. Archives of pathology & laboratory medicine. 137(8): 1141-1146.
24. Wouters et al (2020). Alarmed by misleading interference in free T3 and free T4 assays: a new case of anti-streptavidin antibodies. Clin Chem Lab Med. 58(3): e69-e71.
25. Robier et al. (2020). Anti-streptavidin antibodies as a cause of false-positive results of streptavidin-based autoantibody assays. Clin Chem Lab Med. 58(1):e5-e7.
26. Verougstaete et al. (2019). Interference of anti-streptavidin antibodies in immunoassays:

a very rare phenomenon or a more common finding? Clin Chem Lab Med. 20191064, eISSN 1437-4331, ISSN 1434-6621.
27. U.S. patent application publication no. 2019/0137484
28. Waner M. J., et al. (2019). Streptavidin cooperative allosterism upon binding biotin observed by differential changes in intrinsic fluorescence. Biochem Biophys Rep. 17: 127-131
29. Sano T., Cantor C. R. (1990). Cooperative biotin binding by streptavidin. Electrophoretic behavior and subunit association of streptavidin in the presence of 6 M urea. J Biol Chem. 265(6): 3369-3373.
30. Song, J., et al. (2015). Functional loop dynamics of the streptavidin-biotin complex. Sci Rep. 5: 7906
31. Deng, L., et al. (2013). Dissociation kinetics of the streptavidin-biotin interaction measured using direct electrospray ionization mass spectrometry analysis. J Am Soc Mass Spectrom. 24(1): 49-56.

1. A method for mitigating an interference from a liquid biological sample, the method comprising:
a) combining the sample with a particle comprising streptavidin to provide a mixture;
b) mixing the mixture to facilitate binding of the interference to the streptavidin; and
c) separating the particle from the sample;
thereby removing or reducing the amount of the interference.

2. The method of claim 1, wherein biotin binding sites on the streptavidin are unoccupied so that interference due to anti-streptavidin, biotin, or both are removed or reduced.

3. The method of claim 1, wherein the biotin binding sites on the streptavidin are saturated with biotin so that interference due to anti-streptavidin is removed or reduced.

4. The method of claim 3, wherein the streptavidin is biotinylated so that interference due to anti-streptavidin, anti-biotin, or both are removed or reduced.

5. The method of claim 1, wherein the streptavidin is conjugated with an additional non-biotin capture moiety, so that interference due to a substance that binds the capture moiety is also removed or reduced.

6. The method of claim 1, wherein the particle is magnetic.

7. The method of claim 6, wherein separating the particle from the sample comprises exposing the mixture to a magnet and collecting the liquid sample.

8. A method for mitigating an interference from a liquid biological sample, the method comprising:
a) combining the sample with a biotin-saturated streptavidin (QSAv) to provide a mixture;
b) mixing the mixture to facilitate binding of the interference to the streptavidin;
thereby blocking or reducing the amount of anti-streptavidin interference.

9. The method of claim 8, wherein the QSAv is biotinylated so that interference due to anti-streptavidin, anti-biotin, or both are blocked or reduced.

10. The method of claim 8, wherein the QSAv is conjugated with an additional non-biotin capture moiety, so that interference due to a substance that binds the capture moiety is also blocked or reduced.

11. The method of claim 10, wherein the biotinylation or conjugation is covalent.

12. The method of claim 11, wherein the biotinylation comprises use of an ester-derivatized biotin.

13. The method of claim 12, wherein the ester-derivatized biotin is selected from the group consisting of NHS-biotin, NHS-LC-biotin, NHS-LC-LC-biotin, TFP-LC-biotin, NHS-chromalink-biotin, NHS-PECM-biotin, TFP-(PEO)n-biotin, and NHS-(PEO)n-biotin.

14. The method of claim 10, wherein the biotinylation or conjugation is mediated by a biotin-linker non-covalently bound to a biotin binding site on the streptavidin.

15. The method of claim 6, wherein the QSAv is predominantly monomeric.

16-43. (canceled)
